(19) United States
(12) Patent Application Publication
(10) Pub. No.: US 2016/0231319 A1
(43) Pub. Date: Aug. 11, 2016

(54) METHOD OF ENHANCING HEMATOPOIETIC CELL TRANSPLANTATION

(71) Applicant: David R. KAPLAN, Shaker Heights, OH (US)

(72) Inventor: David R. KAPLAN, Shaker Heights, OH (US)

(73) Assignee: Verve, Ltd., Pepper Pike, OH (US)

(21) Appl. No.: 14/788,171

(22) Filed: Jun. 30, 2015

Related U.S. Application Data
(60) Provisional application No. 62/018,926, filed on Jun. 30, 2014.
(60) Provisional application No. 62/068,998, filed on Oct. 27, 2014.
(60) Provisional application No. 62/069,616, filed on Oct. 28, 2014.

(51) Int. Cl. G01N 33/569; A61K 35/28; C12Q 1/68

(52) U.S. Cl. CPC G01N 33/56966; C12Q 1/6876; A61K 35/28; G01N 2333/47; G01N 2333/948; G01N 2333/91205; G01N 2333/82; C12Q 2600/158; A61K 2035/124

(57) ABSTRACT

The invention relates to a method for enhancing the transplantation of hematopoietic cells to supplement or fully reconstitute the hematopoietic system, such as in myeloablated patients or patients otherwise deficient in hematopoietic cells. The method involves administering CD34⁺ cells having enhanced expression of one or more of AML-1, MYSM1, Hif1a, Profilin-1, phospho-GSK-3beta, SKP2, cbx7, Bmi-1, TCF1, Musashi-2, or FLI1 at levels that provide desirable therapeutically effective amounts of self-renewal of the administered cells and desirable therapeutically effective amounts of differentiation of the administered cells into the various progeny cells of the hematopoietic system (i.e., therapeutically effective amounts of hematopoietic reconstitution). To provide such cells to a subject, the invention relates to detecting such cells prior to or during treatment to ascertain whether such cells are present in clinically-relevant amounts. It may also relate to treating a subject so as to provide clinically-relevant numbers of such cells, as with specific mobilization agents.

### FIELD OF THE INVENTION

The invention specifically relates to a method for enhancing the transplantation of hematopoietic cells to supplement or fully reconstitute the hematopoietic system, such as, in myeloablated patients or patients otherwise deficient in hematopoietic cells. The method involves administering CD34⁺ cells co-expressing one or more of AML-1, MYSM1, Hif1a, NPM-1, Profilin-1, phospho-GSK-3Beta, SKP2, cbx7, Bmi-1, TCF1, Musashi-2, or FLI1 at certain levels to provide self-renewal of the administered cells and/or differentiation of the administered cells into the various progeny cells of the hematopoietic system (i.e., therapeutically effective amounts of hematopoietic reconstitution). To provide such cells to a subject, the invention relates to detecting such cells prior to or during treatment to ascertain whether such cells are present in clinically-relevant numbers. It may also relate to treating a subject so as to provide clinically-relevant numbers of such cells, as with specific mobilization agents. It may also relate to treating a subject with umbilical cord blood cells or with cells that have been cultured to be expanded in numbers or cultured to be enhanced in potency for hematopoietic reconstitution. The invention also relates to compositions containing the cells. The invention generally relates to methods for identifying genes, the expression of which is associated with a desired clinical outcome in the context of cell transplantation, and then using the expression levels of these genes in a sample for cell transplantation as a predictive marker of a clinical outcome.

### BACKGROUND OF THE INVENTION

The hematopoietic system can be reconstituted by cells that are the progenitor/stem cells for all blood cells. These stem/progenitor cells can be designated, as in this application, “hematopoietic-reconstituting cells” or “HRC.” Hematopoietic-reconstituting cells are capable of self-renewal and of differentiating into any cell in the hematopoietic system, including lymphocytes, monocytes, platelets, erythrocytes and myeloid cells. Hematopoietic-reconstituting cells have therapeutic potential as a result of their capacity to restore blood and immune cell function.

Transplantation of CD34⁺ hematopoietic-reconstituting cells is an important treatment modality for malignant and nonmalignant disorders. Most commonly, hematopoietic-reconstituting cells from bone marrow are mobilized into the peripheral blood by pharmacological treatment, thereby facilitating collection. The number of CD34⁺ cells in mobilized blood samples is used to indicate the appropriateness of transplantation although it does not necessarily distinguish between two necessary functions for hematopoietic reconstitution, specifically, long-term reconstitution, mediated by cells with self-renewing proliferation, and short-term hematopoietic differentiation, mediated by progenitor cells.

Transplantation of hematopoietic-reconstituting cells from bone marrow, mobilized peripheral blood and umbilical cord blood has been used to treat hematopoietic cancers such as leukemia and lymphomas, and to aid hematopoietic system recovery from high-dose chemotherapy. Myelosuppression and myeloablation often result from high-dose chemotherapy. Prior to treatment with high-dose chemotherapy, bone marrow hematopoietic progenitor/stem cells can be mobilized into the peripheral blood so that peripheral blood can be harvested and stored for later use as a source of hematopoietic-reconstituting cells. The transplantation of the stored hematopoietic-reconstituting cells can rescue hematopoietic functions after high-dose chemotherapy. Allogeneic or autologous hematopoietic-reconstituting cells can be used to mediate hematopoietic reconstitution.

It would be desirable if hematopoietic-reconstituting cells could be definitively identified in a heterogeneous mixture of cells by assessing the cells for the expression of markers associated with hematopoietic-reconstituting function. The CD34⁺ cell number has been used as a marker for the progenitor/stem cell quantity. However, the CD34 molecule is not associated with the two critical hematopoietic-reconstituting cell functions: the capacity for self-renewing proliferation and short-term differentiation into hematopoietic cells. See Suzuki, A. et al., Blood (1996) 87:3550-3562. Although the number of CD34⁺ cells can be determined, there remains a large variability in predicting hematopoietic reconstitution. It would be desirable if hematopoietic-reconstituting cells could be evaluated for their potency in mediating hematopoietic-reconstituting function by assessing the expression levels of molecules involved in mediating this function.

There are two sources of HRC that have been shown to be superior to bone marrow CD34⁺ cells in terms of their functional potency on a cell-by-cell basis. They are granulocyte colony-stimulating factor (G-CSF)-mobilized CD34⁺ cells obtained from the peripheral circulation and umbilical cord blood CD34⁺ cells. For instance, at the Fred Hutchinson Cancer Research Center in Seattle, G-CSF-mobilized cells demonstrate 5-7 days faster reconstitution compared to bone marrow cells even when similar doses of CD34⁺ cells were used (Heimfeld, S. Leukemia (2003) 17:856-858.). Enhancement in the recovery of neutrophils (7 days) and platelets (8 days) after transplantation of similar numbers of mobilized peripheral blood CD34+ cells versus bone marrow CD34⁺ cells was also observed in a Norwegian study (Heldal D, et al. Bone Marrow Transplant (2000) 25:1129-1136.). These results are consistent with the greater number of granulocyte-macrophage colony-forming units per CD34⁺ cell for G-CSF mobilized peripheral blood CD34⁺ cells compared to bone marrow resident CD34⁺ cells (Pavletic Z S, et al. J Clin Oncol (1997) 15:1608-1616.).

Similarly, umbilical cord blood HRC have been shown to have a higher cloning efficiency, to proliferate more rapidly in response to cytokine stimulations, and to generate about 7-fold more progeny than HRC from the adult bone marrow (Hao Q-L, et al. Blood (1995) 86:3745-3753.). Another group of investigators found that cultures of cord blood cells produced a significantly greater increase in granulocyte-macrophage colony-forming units and granulocyte-erythrocyte-monocyte-megakaryocyte colony-forming units than cultures of bone marrow cells (Broxmeyer H E, et al. Proc. Natl. Acad. Sci. USA (1992) 89:4109-4113.). A third group found similar findings in comparing umbilical cord blood CD34⁺ cells with bone marrow CD34⁺ cells (Cardoso A A, et al. Proc. Natl. Acad. Sci. USA (1993) 90:8707-8711.). It is also important to note that approximately 10-fold less umbilical cord blood CD34 cells are used for transplantation than the bone marrow CD34⁺ cells.

### SUMMARY OF THE INVENTION

The inventor has discovered that one can predict the potency of a sample of CD34⁺ cells to reconstitute the hematopoietic system by assessing the expression levels of one or more of AML-1, MYSM1, Hif1a, NPM-1, Profilin-1, phospho-GSK-3Beta, SKP2, cbx7, Bmi-1, TCF1, Musashi-2, or FLI1 in the CD34⁺ cells in the sample. The inventor has assessed the expression levels of these molecules in CD34⁺ cells from various different subject groups, including, bone marrow from healthy subjects, umbilical cord blood, and mobilized blood from healthy subjects. It is known and accepted that CD34⁺cells from either umbilical cord blood or from healthy subjects pharmacologically treated to mobilize their cells from the bone marrow are superior in hematopoietic-reconstituting function to CD34⁺ cells from the bone marrow of healthy persons. The inventor discovered that certain expression levels of AML-1, MYSM1, Hif1a, NPM-1, Profilin-1, phospho-GSK-3Beta, SKP2, cbx7, Bmi-1, TCF1, Musashi-2, or FLI1 in CD34⁺ cells are associated with the sources having greater functional potency as assessed engraftment time. The inventor has found that the expression of these molecules in a sample of CD34⁺ cells can be used to predict the actual clinical outcome of treatment by transplantation of the sample to a patient.

Accordingly, specific expression levels of one or more of the molecules in CD34⁺ cells (i.e., AML-1, MYSM1, Hif1a, NPM-1, Profilin-1, phosphor-GSK-3Beta, SKP2, cbx7, Bmi-1, TCF1, Musashi-2, or FLI1) can be used to recognize potency of a sample in terms of hematopoietic reconstitution. Furthermore, based on these findings, potency of CD34⁺ cells in a subject can be manipulated by the addition of specific agents, such as, mobilization agents to the patient or by culturing cells with or without specific agents that increase or decrease the expression of these genes in the CD34⁺ cells, and thus increase potency of the sample. Thus, therapeutically-effective amounts of cells with desired expression of one or more of the molecules can be recognized.

“Enhanced or decreased expression” is expression compared to the median or mean level of expression from a sample of about 20 or more specimens of the same origin and type (for example, cells from bone marrow from a subject that has been treated with a mobilizing agent). These more potent samples now can be obtained and administered to a subject to improve reconstitution of the hematopoietic system.

The invention is also directed to a method to identify a molecule, the expression of which is correlated with hematopoietic reconstituting function, the method comprising assessing expression of the molecule in individual CD34⁺ cells in samples having different levels of potency and identifying molecules, the expression of which correlates with potency, by correlating differences in expression of such molecules with the potency of the different samples. The molecule can then be used as a predictor of potency in a treatment sample by corroborating its expression (or lack thereof) in samples that can be associated with real clinical outcomes (e.g. time to engraftment).

The expression level of a single molecule can be related to enhanced reconstitution by simple linear regression; however, the invention is also directed to the use of other statistical analyses more appropriate for assessing the predictive value of the expressions of multiple molecules on enhanced reconstitution. These other statistical analyses include multiple linear regression, linear regression models including but not limited to factor analysis and principal component analysis, and nonlinear regression models including but not limited to neural networks, K-nearest neighbor analysis, support vector machines, and multiple adaptive regression splines. Linear and nonlinear classification models can also be used to stratify samples based on levels of expression of more than 1 molecule. Classification models include, but are not limited to, discriminant analysis, hierarchical clustering, logistic regression, naïve Bayes nonlinear classification, and classification trees. In this regard, this application incorporates U.S. Ser. No. 13/829,557 by reference for the statistical methods and their application for correlating gene expression with potency.

Greater potency can be associated with an increase or decrease in expression, depending on the gene. In one embodiment AML-1, MYSM1, Hif1a, NPM-1, Profilin-1, phospho-GSK-3Beta, SKP2, cbx7, Bmi-1, TCF1, Musashi-2, or FLI1 are found at enhanced levels in the more potent samples.

Samples that are compared can be bone marrow, mobilized peripheral blood from healthy or diseased subjects, and umbilical cord blood.

The expression that is assayed can be selected from the group consisting of RNA, protein, and post-translational modification.

The method can be used to assess expression of molecules and pathways associated with HRC function, which can include the following: Notch pathway, nucleoside salvage pathway, OTT-1, MEIS1, Ap2a2, Lin28b, Wnt signaling pathway, MetAP2 (methionine aminopeptidase 2), Pot1b, Evi1, Smad signaling, Erg (E-26-related gene), PCNA (proliferating cell nuclear antigen associated factor), Rac1/Rac2/Rac3, Prdm16, APC, Rho GTPase, p190-B, Fbw7, Gli1, Ldb1, NKAP, cyclin C, Irgm1, HoxA9, NA10HD, Fbxw7alpha, IRF8, NUP98, MycN, DDX10, ANGPT1, REN, HEY1, Sox4, Stat5, Slug, p53, prostaglandin E2, Zfx, Calcineurin, NFAT, cyclin E2, SHIP, NF-Y, Hedgehog pathway, Dmtf1, Nrf2, ANKRD28, GNA15, UGP2, Skp2, Mdm2, Sox7, Ikaros, TET2, SCL, TAL1, Jumonji, Lyl1, Foxo3a, Gimap5, ADAR1, Menin, Wnt3a, PSF1, ABCG2, Tie1/2, cMp1, CD117, mTORC1, c-Cbl, Rb, Pbx1, EWS, PU.1, Chk1, Necdin, SHP2, PUMA, FUS, WASP, NOD2, Mef2c, GABP, Angptls, SIRT1, 12/15-lipoxygenase-dependent fatty acid metabolism pathway, angiopoietin-1, angiopoietin-2, Cited2, SIMPL, p300, Heme oxygenase-1, p16ink4A, p18ink4c, p21cip1, Survivin. Frizzled-related protein 1, Rheb2, aldehyde dehydrogenase 1a1, CD130, CD123.

The invention, in a more general form, is measuring the expression level of a molecule or expression levels of molecules in cells to be transplanted for therapeutic purposes in order to assess the relative potency of the cellular inoculum in terms of a desired clinical, functional, or therapeutic outcome.

Measuring the expression level of a molecule or molecules can be accomplished by a variety of methods, including, flow cytometry, western analysis, mass spectroscopy, immunoassay, northern analysis, nucleic acid arrays, or nucleic acid amplification procedures, such as, PCR. Expression is assessed on a per cell basis and may be assessed in individual cells.

Cells are transplanted as a therapeutic procedure for many indications. For instance, the transplantation of hematopoietic stem cells has been used for over 30 years to reconstitute hematopoiesis in patients treated with chemotherapeutic agents to kill cancer cells. Other types of cells that can be transplanted for therapeutic purposes include, but are not limited to, mesenchymal stem cells to mediate immunosuppression for patients with graft-versus-host disease or multiple sclerosis or inflammatory bowel disease; T lymphocytes expressing chimeric antigenic receptors as a treatment of cancer; dendritic cells to vaccinate patients; mesenchymal stem cells to treat joint disease; hematopoietic stem cells to recover cardiac function after myocardial infarction; and embryonic stem cells or induced pluripotent stem cells to treat eye diseases or neurological diseases.

The desired therapeutic outcome is understood in cellular transplantation. The inoculation of hematopoietic stem cells after chemotherapeutic intervention can be performed in order to reconstitute hematopoiesis. Mesenchymal stem cells can be transplanted to suppress immunity for patients with autoimmune disease. T lymphocytes expressing chimeric antigenic receptors can be used to kill cancer cells. Dendritic cells can be transplanted to induce a powerful immune response in the recipient.

The expression level of any molecule or set of molecules can be measured in cells. In a preferred embodiment the molecules are chosen for their known relevance to the therapeutic outcome desired. For example, expression levels of molecules known to be involved in hematopoietic reconstitution can be measured in hematopoietic stem cells and molecules known to be functional in antigenic processing and presentation can be assessed in dendritic cells.

Cells transplanted for therapeutic purposes can be relatively enriched for the function required. For example, dendritic cells make up 70% or more of the cells transplanted for the purpose of inducing an immune response. Alternatively, the function required may reside in a small proportion of the cells transplanted. Hematopoietic stem cells usually make up less than 5% of the cells transplanted in order to reconstitute hematopoiesis (for example, in UCB). The expression levels of informative molecules may be measured in all of the cells transplanted or in a subpopulation of the cells transplanted. The levels are determined on a per cell basis and may be determined in individual cells.

Potency measures are desirable for cellular transplantation. The FDA has asked for potency measures for cells transplanted for therapeutic purposes. Potency measures are most useful if they indicate the probability of the cells effecting the desired clinical outcome. Consequently, the invention is efficacious because it provides an increased probability of obtaining a desired clinical effect.

The invention also contemplates cell banks of autologous or allogeneic samples of desired (known or unknown) potency to be used as an “off the shelf” source of cells for transplantation.

Accordingly, after expression of a gene is established as an adequate predictor of clinical outcome/potency, any sample of unknown potency can be tested for expression of that gene prior to transplantation into a patient. For example, the process may be as follows: (1) from about 20 or more samples (e.g. UCB), establish the mean or median expression of gene; (2) assess expression of the gene compared to the mean/median in samples of known potency (look for significant deviation); (3) assess the expression levels of the gene in a sample to be transplanted; (3) compare the levels in that sample with the mean/median levels; and (4) use the sample for treatment if the level correlates with adequate potency.

### BRIEF DESCRIPTION OF THE DRAWINGS

FIG. 1 is a schematic representation of hematopoietic differentiation.

FIG. 2 is a flow chart illustrating a method for preparing a subject for donating blood in accordance with an embodiment of the present invention.

FIG. 3 is Table 1 which shows the principal component analysis of the expression levels of 6 analytes and the engraftment time as indicated by the number of days to a specified threshold.

FIG. 4 is Table 2 which shows cluster analysis including expression levels of 6 analytes.

### DETAILED DESCRIPTION OF THE INVENTION

### Definitions

“A” or “an” means herein one or more than one; at least one. Where the plural form is used herein, it generally includes the singular.

The term “AML-1” is understood to refer to acute myeloid leukemia 1 protein or RUNX1 which is runt-related transcription factor 1, encoded by a gene having, in humans, the sequence shown in NCBI Reference Sequence: locus AAI36381. The sequence can be found at the following site: http://www.ncbi.nlm.nih.gov/protein/AA136381.1 incorporated by reference for the sequence. The amino acid sequence coding for AML-1 can also be found at SEQ ID: 1; and its corresponding nucleotide sequence can be found at SEQ ID: 2. This gene may, like most other genes, contain polymorphisms that still allow the gene to maintain the function. With respect to this application, it would be sufficient function so as to provide clinically-relevant levels of cells for hematopoietic reconstitution or other transplantation. The gene also includes, for non-human uses, such as veterinary uses, orthologs from other mammals. These include companion animals, farm animals and sport animals, for example, felines, canines, bovines, equines, porcines, ovines, etc.

Bmi-1, known as B cell-specific Moloney murine leukemia virus integration site 1, a polycomb complex protein, also known as polycomb group RING finger protein 4; locus NP_005171; http://www.ncbi.nlmn.nih.gov/protein/NP_005171.4. Amino acids coding for Bmi-1 can also be found at SEQ ID: 19, while its corresponding nucleotide sequence can be found at SEQ ID: 20.

cbx7, known as chromobox protein homolog 1; locus CAG33047; http://www.nchi.nlm.nih.gov/protein/NP_783640.1. Amino acid sequences coding for cbx7 can also be found at SEQ ID: 17, while its corresponding nucleotide sequence can be found at SEQ ID: 18.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration to the subject, it may be preferable to again assay the cells for potency. This can be done using any of the assays, direct or indirect, described in this application or otherwise known in the art. Then cells having the desired potency can then be administered to the subject for treatment. Banks can be made using cells derived from the individual to be treated (from their pre-natal tissues such as placenta, umbilical cord blood, or umbilical cord matrix or expanded from the individual at any time after birth). Or banks can contain cells for allogeneic uses.

A “clinical outcome” refers to a physical or mental effect in a patient that demonstrates effective treatment. See also a “therapeutically effective amount” below. This may also be manifested by primary physical effects, such as, engraftment.

Also, cells can be grown in culture and used for transplantation. For instance, pluripotent stem cells or embryonic stem cells have been differentiated to hematopoietic-reconstituting cells in culture.

“Co-administer” means to administer in conjunction with one another, together, coordinately, including simultaneous or sequential administration of two or more agents. In the context of the invention, the two types of CD34⁺ cells can be administered with these alternative regimens.

“Comprised of” is a synonym of “comprising”.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Decrease” or “reduce” means to lack entirely as well as to contain/express in lower amounts.

“Desired expression” refers to an enhanced or decreased expression level, whichever is associated with the desired clinical outcome.

“Effective amount” generally means an amount which provides the desired effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.” In the context of the invention, effective amounts are amounts of those CD34⁺ cells with enhanced expression of one or more of AML-1, MYSM1, Hif1a, NPM-1, Profilin-1, phospho-GSK-3beta, SKP2, cbx7, BMi-1, TCF1, Musashi-2, or FLI1 that provide clinically-significant hematopoietic reconstitution (i.e., potency). “Effective expression” refers to expression that provides for that clinically-significant reconstitution.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

The term “enhanced”, as it is applied to the invention, means expression of one or more of AML-1, MYSM1, Hif1a, NPM-1, Profilin-1, phospho-GSK-3beta, SKP2, cbx7, BMi-1, TCF1, Musashi-2, or FLI1 that is greater than the mean or median expression of those molecules (RNA and/or protein) in a sample of 20 or more specimens of the same origin and type.

Enhanced or decreased expression is expression compared to 20 or more specimens of the same origin and type. As an example, to assess the potency of a sample to be used for treatment (e.g., UCB) and predict successful engraftment, one would assess at least 20 samples of UCB for expression of the particular gene. One would determine the mean or median level of expression per cell. Then one would test the sample for expression of the gene that is significantly above or below the mean or median level. That gene, thus, is a predictor of actual clinical outcome, e.g., engraftment.

Determining the level of expression of a gene or genes refers to levels to a level of expression on a per cell basis. This can be determined in individual cells, for example, where the relevant cell is found in a heterogeneous mixture (such as HRCs in whole UCB). Or the level can be determined by measuring expression in a population (such as a homogeneous population).

One could find out if a gene is a good predictor as follows: One would obtain samples of cells that have actually been transplanted and that provided a clinical outcome. Then one would determine if expression of the candidate gene is significantly associated with the outcome. If it is then it can be used to predict the efficacy of samples used in the future.

The term “FLI-1” is understood to refer to Friend leukemia integration 1 transcription factor also known as transcription factor ERGB, encoded by a gene having, in humans, the sequence shown in NCBI Reference Sequence: locus AAA58480. The sequence can be found at the following site: http://www.ncbi.nim.nih.gov/protein/AAA58480.1 incorporated by reference for the sequence. The coding amino acid sequence coding for FLI-1 can also be found at SEQ ID: 3, while its corresponding nucleotide sequence can be found at SEQ ID: 4. This gene may, like most other genes, contain polymorphisms that still allow the gene to maintain the function. With respect to this application, it would be sufficient function so as to provide clinically-relevant levels of cells for hematopoietic reconstitution or other transplantation. The gene also includes, for non-human uses, such as veterinary uses, orthologs from other mammals. These include companion animals, farm animals and sport animals, for example, felines, canines, bovines, equines, porcines, ovines, etc.

The term “hematopoietic-reconstituting cell” or “HRC”, as used herein, refers to a progenitor and/or stem cell that can reconstitute all of the hematopoietic cells in a subject. These include, but are not limited to, lymphocytes, platelets, erythrocytes and myeloid cells, including, T cells, B cells (plasma cells), natural killer cells, dendritic cells, monocytes (macrophages), neutrophils, eosinophils, basophils (mast cells), megakaryocytes (platelets), and erythroblasts (erythrocytes). The HRC are also capable, in addition to differentiation, of self-renewal, so as to proliferate the stem-progenitor population that is capable of differentiation.

The term “hematopoietic-reconstituting cell” or “HRC” generally refers to the functions of the cells that provide their ability to reconstitute the hematopoietic system to provide a clinically-relevant effect. Technically, the reconstitution function can be broken down into two functions that may be represented by two sets of cells: (1) CD34⁺ self-renewing hematopoietic-reconstituting cells and (2) CD34⁺hematopoietic-reconstituting cells that differentiate into hematopoietic cell progeny. See pending U.S. patent application Ser. No. 13/490,000, incorporated by reference for disclosure of these cells.

The term “Hif1a” is understood to refer to hypoxia-inducible factor-1-alpha, encoded by a gene having, in humans, the sequence shown in NCBI Reference Sequence: locus NP 851397. The sequence can be found at the following site: http://www.ncbi.nlm.nih.gov/protein/NP_851397.1 incorporated by reference for the sequence. The amino acid sequence coding for Hif1a can also be found at SEQ ID: 5, while its corresponding nucleotide sequence can be found at SEQ ID: 6. This gene may, like most other genes, contain polymorphisms that still allow the gene to maintain the function. With respect to this application, it would be sufficient function so as to provide clinically-relevant levels of cells for hematopoietic reconstitution or other transplantation. The gene also includes, for non-human uses, such as veterinary uses, orthologs from other mammals. These include companion animals, farm animals and sport animals, for example, felines, canines, bovines, equines, porcines, ovines, etc.

Use of the term “includes” is not intended to be limiting.

“Increase” or “increasing” means to induce entirely, where there was no pre-existing effect, as well as to increase the degree.

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only hematopoietic-reconstituting cells. Rather, the term “isolated” indicates that the cells are removed from their natural tissue environment and are present at a higher concentration as compared to the normal tissue environment. Accordingly, an “isolated” cell population may further include cell types in addition to hematopoietic-reconstituting cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (for example, bone marrow, peripheral blood, umbilical cord blood, etc.).

Musashi-2, is an RNA-binding protein; locus NP_620412; http://www.ncbi.nlm.nih.gov/protein/NP_620412.1. Amino acids coding for Musashi-2 can also be found at SEQ ID: 23, while its corresponding nucleotide sequence can be found at SEQ ID: 24.

The term “MYSM1” is understood to refer to a metalloprotease that specifically deubiquitinantes monobiguitinated histone H2A, Myb-Like, SWIRM and MPN domain-containing protein 1, encoded by a gene having, in humans, the sequence shown in NCBI Reference Sequence: locus NP 001078956. The sequence can be found at the following site: http://www.ncbi.nln.nig.gov/_protein/NP_001078956.1 incorporated by reference for the sequence. Amino acids coding for MYSM1 can also be found at SEQ ID: 7, while its corresponding nucleotide sequence can be found at SEQ ID: 8. This gene may, like most other genes, contain polymorphisms that still allow the gene to maintain the function. With respect to this application, it would be sufficient function so as to provide clinically-relevant levels of cells for hematopoietic reconstitution or other transplantation. The gene also includes, for non-human uses, such as veterinary uses, orthologs from other mammals. These include companion animals, farm animals and sport animals, for example, felines, canines, bovines, equines, porcines, ovines, etc.

The term “NPM-1” is understood to refer to nucleophosmin or nucleolar phosphoprotein B23 or numatrin, encoded by a gene having, in humans, the sequence shown in NCBI Reference Sequence: locus AAH09623. The sequence can be found at the following site: http://www.ncbi.nlm.nih.gov/protein/AAH09623.1 incorporated by reference for the sequence. Amino acids coding for NPM-1 can also be found at SEQ ID: 9, while its corresponding nucleotide sequence can be found at SEQ ID: 10. This gene may, like most other genes, contain polymorphisms that still allow the gene to maintain the function. With respect to this application, it would be sufficient function so as to provide clinically-relevant levels of cells for hematopoietic reconstitution or other transplantation. The gene also includes, for non-human uses, such as veterinary uses, orthologs from other mammals. These include companion animals, farm animals and sport animals, for example, felines, canines, bovines, equines, porcines, ovines, etc.

“Pharmaceutically-acceptable carrier” is any pharmaceutically-acceptable medium for the cells used in the present invention. Such a medium may retain isotonicity, cell metabolism, pH, and the like. It is compatible with administration to a subject in vivo, and can be used, therefore, for cell delivery and treatment.

Phospho-GSK-3beta, refers to glycogen synthase kinase-3 phosphorylated on serine at the 9 amino acid position; locus NP_002084 http://www.ncbi.nlm.nih.gov/protein/NP_002084.2. Amino acids coding for GSK-3beta can also be found at SEQ ID: 13, while its corresponding nucleotide sequence can be found at SEQ ID: 14.

The term “potency” may refer to the degree of the ability of a cell population to provide hematopoietic-reconstituting cell effects, i.e., self-renewal and/or differentiation, sufficient to achieve a clinically-detectable result. In a specific context of the invention, potency refers to the numbers of CD34⁺ cells having desired expression of one or more of the genes, i.e., that provide greater potency to the sample. However, “potency” more broadly refers to the ability of a cellular sample to provide a desired clinical outcome.

Profilin-1, is a small actin-binding protein that regulates actin polymerization; locus NP_005013; http://www.ncbi.nlm.nih.gov/protein/NP_005013.1. Amino acids coding for Profilin-1 can also be found at SEQ ID: 11, while its corresponding nucleotide sequence can be found at SEQ ID: 12.

The term “reconstitute” implies a range of increase from a fully or partially deficient hematopoietic system. It is not limited to, for example, cases in which the entire hematopoietic system is ablated. Reduced intensity conditioning is used in HRC transplantation. Reduced intensity conditioning does not result in myeloablation and it is used in patients that are older, in patients who are in complete remission, and in patients with acquired aplastic anemia.

The term “reduce” as used herein means to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject. This also applies to the biological effects such as self-renewal and differentiation.

“Selecting” a cell with a desired level of potency can mean identifying (as by assay), isolating, and expanding a cell. This could create a population that has a higher potency than the parent cell population from which the cell was isolated.

To select a cell could include both an assay to determine if there is the desired effect and could also include obtaining that cell. The cell may naturally have the effect in that the cell was not incubated with or exposed to an agent that induces the effect. The cell may not be known to have the effect prior to conducting the assay. As the effects could depend on gene expression and/or secretion, one could also select on the basis of one or more of the genes that cause the effects.

Selection could be from cells in a tissue, e.g., UCB. Selection could be directly from the tissue or from cultured cells. For example, in this case, cells could be isolated from a desired tissue, expanded in culture, selected for a desired effect, and the selected cells further expanded.

Selection could also be from cells ex vivo, such as cells in culture. In this case, one or more of the cells in culture would be assayed for the effect and the cells obtained that have the effect could be further expanded.

Cells could also be selected for enhanced effect. In this case, the cell population from which the enhanced cell is obtained already has the effect. Enhanced effectiveness means a higher average amount of the effect per cell than in the parent population.

The parent population from which the enhanced cell is selected may be substantially homogeneous (the same cell type). One way to obtain such an enhanced cell from this population is to create single cells or cell pools and assay those cells or cell pools for the effect to obtain clones that naturally have the effect (as opposed to treating the cells with a modulator of the effect) and then expanding those cells that are naturally enhanced.

However, cells may be treated with one or more agents that will enhance the effect of endogenous cellular pathways. Thus, substantially homogeneous populations may be treated to enhance modulation.

If the population is not substantially homogeneous, then, it is preferable that the parental cell population to be treated contains at least 100 of the effective cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of the effect may be those that are higher than the levels in a given preceding population. For example, cells that are put into primary culture from a tissue and expanded and isolated by culture conditions that are not specifically designed to have the effect, may provide a parent population. Such a parent population can be treated to enhance the average effect per cell or screened for a cell or cells within the population that express higher effect. Such cells can be expanded then to provide a population with a higher (desired) effect.

Whereas the exemplified hematopoietic-reconstituting cells in this application express the genes naturally (i.e., not by recombinant means, such as by exogenous promoter/enhancer insertion into the endogenous gene, or by the addition of exogenous coding sequences), the invention could cover cells that are genetically engineered for enhanced expression of the genes (for example, by increasing the copy number, reducing the copy number, increasing transcription/translation, or decreasing expression, such as by negative regulators such as small molecules, anti-sense RNA and the like).

“Self-renewal” refers to the ability to produce replicate daughter stem cells having differentiation potential that is identical to those from which they arose. A similar term used in this context is “proliferation.”

SKP2, known as S-phasse kinase-associated protein 2; locus NP_005974 http://www.ncbi.nlm.nih.gov/protein/NP_005974.2. Amino acids coding for SKP2 can also be found at SEQ ID: 15, while its corresponding nucleotide sequence can be found at SEQ ID: 16.

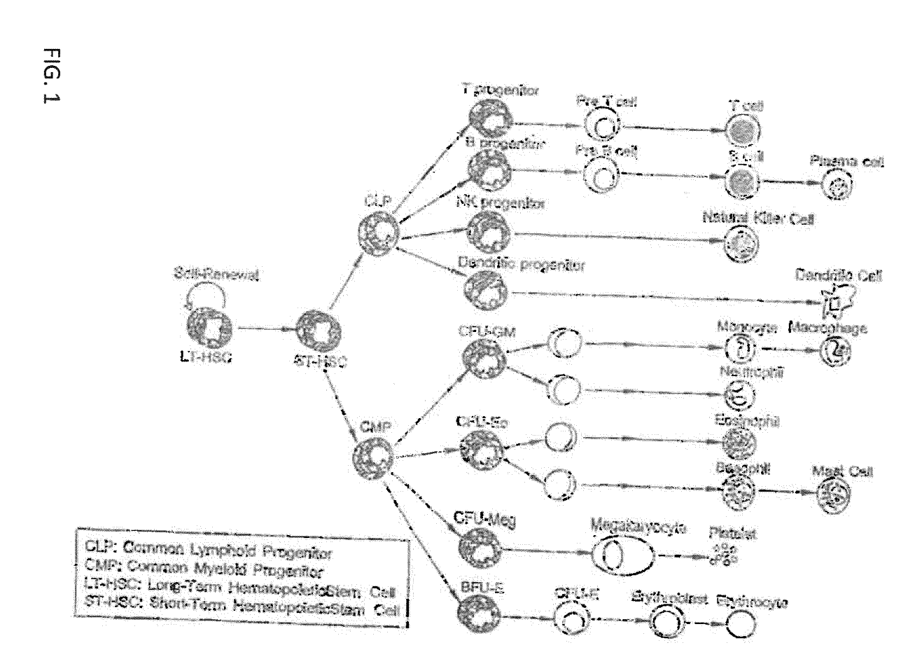

“Stem cell” means a cell that can undergo self-renewal (i.e., progeny with the same differentiation potential) and also produce progeny cells that are more restricted in differentiation potential. In the context of the present invention, differentiation is into hematopoietic progeny, such as shown in FIG. 1.

“Subject” means a vertebrate, such as a mammal, such as a human. Mammals include, but are not limited to, humans, dogs, cats, horses, cows, and pigs.

“Substantially homogeneous” refers to cell preparations where the cell type is of significant purity of at least 50%. The range of homogeneity may, however, be up to and including 100%. Accordingly, the range includes about 50% to 60%, about 60% to 70%, about 70% to 80%, about 80% to 90% and about 90% to 100%. This is opposed to the use of the term “isolated”, which can refer to levels that are substantially less. However, as used herein, the term “isolated” refers to preparations in which the cells are found in numbers sufficient to exert a clinically-relevant biological effect, as described in this application (i.e., transplantation, such as hematopoietic reconstitution).

TCF1, is a transcription factor known as T cell factor 1; locus AAF00616 http://www.ncbi.nlm.nih.gov/protein/AAFX00616.1. Amino acids coding for TCF1 can also be found at SEQ ID: 21, while its corresponding nucleotide sequence can be found at SEQ ID: 22.

The term “therapeutically effective amount” refers to the amount of an agent determined to produce any therapeutic response in a mammal. For example, effective amounts of hematopoietic-reconstituting cells may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of hematopoietic deficiency.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Validate” means to confirm. In the context of the invention, one confirms that a cell is an expressor with a desired potency. This is so that one can then use that cell (in treatment, banking, drug screening, etc.) with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

The cells of the invention can be used to treat various cancers and immune system disorders, including acute myeloid leukemia, chronic myeloid leukemia, acute lymphocytic leukemia, childhood leukemias, myelodysplastic syndromes, multiple myeloma, lymphoma, chronic lymphocytic leukemia, solid tumors in children, breast cancer, solid tumor in adults, germ cell tumors, primary immunodeficiency diseases, Fanconi anemia, acquired aplastic anemia, acquired immunodeficiency diseases, thalessemia, sickle cell anemia, lysosomal storage disorders and autoimmune diseases. This treatment is also used for multiple sclerosis, systemic sclerosis, rheumatoid arthritis, juvenile idiopathic arthritis, systemic lupus erythematosis, and Crohn's disease which are all included under the autoimmune disease heading. Additionally, HRC transplantation (autologous) is used in the treatment of cardiovascular disease and stroke.

### Embodiments of the Invention

In one embodiment the invention is directed to a method for assessing the capacity of a sample to therapeutically effect hematopoietic reconstitution in a subject, the method comprising assessing individual CD34⁺ cells for desired expression of one or more of AML-1, MYSM1, Hif1a, NPM-1, Profilin-1, phospho-GSK-3beta, SKP2, cbx7, Bmi-1, TCF1, Musashi-2, or FLI1, in the sample. One may also determine the number of those cells to verify that there are a sufficient number to effect the desired clinical outcome (e.g., engraftment).

In particular, the levels of one or more of AML-1, MYSM1, Hif1a, NPM-1, Profilin-1, phospho-GSK-3beta, SKP2, cbx7, Bmi-1, TCF1, Musashi-2, or FLI1 in these CD34⁺ cells is assessed. The assessment is for cells that express the one or more of AML-1, MYSM1, Hif1a, NPM-1, Profilin-1, phospho-GSK-3beta, SKP2, cbx7, Bmi-1, TCF1, Musashi-2, or FLI1 at levels greater or less than the mean or median expression in a sample of 20 or more specimens of the same origin and type.

The number of these cells provide useful predictors of the effectiveness of a sample from any given tissue source. Accordingly, if a sample is selected from a particular source and assessed for numbers of cells with the desired expression and found to have numbers that are too low to be effective, this sample may be found unsuitable for transplantation.

In one embodiment the invention is directed to a method to therapeutically effect hematopoietic reconstitution in a subject, the method comprising administering to a subject an agent that provides desired expression of one or more of AML-1, MYSM1, Hif1a, NPM-1, Profilin-1, phospho-GSK-3beta, SKP2, cbx7, Bmi-1, TCF1, Musashi-2, or FLI1 in CD34⁺ cells in the subject so as to provide a therapeutically-effective amount of cells that effect therapeutic levels of reconstitution.

In one embodiment the invention is directed to a method to prepare a subject to donate blood for hematopoietic-reconstituting cell transplantation, the method comprising obtaining a blood sample containing hematopoietic cells from a subject who has been given a mobilizing agent; determining the expression levels of one or more of AML-1, MYSM1, Hif1a, NPM-1, Profilin-1, phospho-GSK-3beta, SKP2, cbx7, Bmi-1, TCF1, Musashi-2, or FLI1 in individual CD34⁺ cells from the blood sample; and then further administering to the subject a mobilizing agent if it is determined that the mobilized blood sample does not contain a therapeutically-desirable amount of CD34⁺ cells expressing desired levels of one or more of AML-1, MYSM1, Hif1a, NPM-1, Profilin-1, phospho-GSK-3beta, SKP2, cbx7, Bmi-1, TCF1, Musashi-2, or FLI1 for desired levels of hematopoietic reconstitution.

In one embodiment the agent increases expression of one or more of AML-1, MYSM1, Hif1a, NPM-1, Profilin-1, phospho-GSK-3beta, SKP2, cbx7, Bmi-1, TCF1, Musashi-2, or FLI1. Expression includes protein, RNA, or protein modification (see below).

In one embodiment the invention is directed to the methods wherein the sample is obtained from blood.

In one embodiment the blood is “mobilized peripheral blood”, that is, peripheral blood from persons treated with agents to effect the mobilization of HRC from the bone marrow into the peripheral circulation.

In one embodiment the blood is umbilical cord blood.

In one embodiment the invention is directed to the methods wherein the sample is from bone marrow.

In one embodiment protein expression is assayed. Protein expression that is assayed can be intracellular, extracellular (i.e. surface), or both.

In another embodiment gene expression is assayed via expression of RNA. RNA can be any RNA, including, messenger RNA and smaller RNA molecules, such as microRNAs.

In a further embodiment, post-translational modifications may be assayed, including phosphorylation, acetylation, nitrosylation, ubiquitination, sulfation, glycosylation, myristoylation, palmistoylation, isoprenylation, farnesylation, geranylgeranylation, alkylation, amidation, acylation, oxidation, SUMOylation, pupylation, neddylation, biotinylation, pegylation, succinylation, selenoylation, citrullination, deamidation, ADP-ribosylation, iodination, hydroxylation, gamma-carboxylation, carbamylation, S-nitrosylation, S-glutathionylation, and malonylation, as well as any other post-translational modification.

In one embodiment gene expression is assessed by flow cytometry. Another embodiment involves the detection of molecular expression levels in enriched cells by western blotting. Another embodiment involves the detection of molecular expression levels via reverse phase protein arrays involving purified cells. Kornblau S et al. Blood 2009: 113:154-164. Immunoassays on lysates of purified or enriched cells is another embodiment. Gene expression can also be assessed by measuring mRNA. mRNA determinations can be obtained with real-time PCR.

In another embodiment gene expression is assessed in single cells.

In another embodiment gene expression assessment is assessed by EAS. EAS® is an amplification technology disclosed in, for example U.S. Pat. Nos. 6,280,961, 6,335,173, and 6,828,109.

In one embodiment the invention is directed to the above methods comprising the step of administering a mobilizing agent to the subject prior to the step of obtaining a blood sample.

In one embodiment the invention is directed to a method for transplanting hematopoietic-reconstituting cells in a subject in need thereof, the method comprising administering to the subject nucleated blood cells comprising a therapeutically-effective amount of CD34⁺ cells having desired expression of one or more of AML-1, MYSM1, Hif1a, NPM-1, Profilin-1, phospho-GSK-3beta, SKP2, cbx7, Bmi-1, TCF1, Musashi-2, or FLI1.

In one embodiment the invention is directed to the above methods wherein the subject has undergone myeloablation.

The invention is directed to the methods herein wherein the subject has a hematopoietic deficiency or malignancy.

In one embodiment the invention is directed to the above methods wherein assessing the co-expression of CD34⁺ and one or more of AML-1, MYSM1, Hif1a, NPM-1, Profilin-1, phospho-GSK-3beta, SKP2, cbx7, Bmi-1, TCF1, Musashi-2, or FLI1 is performed by flow cytometry.

In one embodiment the invention is directed to the above methods wherein the CD34⁺ cells having desired expression of one or more of AML-1, MYSM1, Hif1a, NPM-1, Profilin-1, phospho-GSK-3beta, SKP2, cbx7, Bmi-1, TCF1, Musashi-2, or FLI1 are isolated.

In one embodiment the isolated cells are expanded in culture for future administration. They may be stored as a cell bank.

In one embodiment the invention is directed to the above methods wherein the subject has a disorder treatable by hematopoietic stem cell transplantation.

In one embodiment the invention is directed to the above methods wherein the disorder is a hematopoietic deficiency or malignancy.

In one embodiment, transplantation is with autologous hematopoietic-reconstituting cells. In another embodiment, transplantation is with allogeneic hematopoietic-reconstituting cells.

Various techniques for assessing expression of one or more of AML-1, MYSM1, Hif1a, Profilin-1, phospho-GSK-3beta, SKP2, cbx7, Bmi-1, TCF1, Musashi-2, or FLI1 in CD34⁺ cells that may be used include, but are not limited to, flow cytometry, flow cytometry with tyramide deposition technology (EAS®), single-cell mass cytometry, immunohistochemistry, western analysis after CD34⁺ cell isolation, enzyme-linked immunosorbent assays (ELISA), and nucleic acid analysis including single-cell polymerase chain reaction (PCR).

In one embodiment, the levels of gene expression are assessed by EAS®, disclosed, for example, in U.S. Pat. Nos. 6,280,961, 6,335,173, and 6,828,109, incorporated by reference for the amplification methods disclosed.

Of course these techniques can be generally applied to expression of any desired gene in any desired cell sample.

The CD34⁺ cells may be obtained from bone marrow, umbilical cord blood or peripheral blood. In peripheral blood, CD34⁺ cells occur naturally and can be mobilized from the bone marrow by pharmacological treatment.

With respect to measuring increased versus decreased expression in comparison to the mean or median of expression from 20 samples of the same origin/type, the invention also contemplates the use of standardized beads with specific levels of fluorescence intensity that can be used to assess the level of expression. In this case, beads with standardized levels of fluorescence would be used to assess the level of expression of a given sample. The standardized beads would still be pegged to the distribution of expression among samples of the same origin/type. The standardized fluorescent beads would simply be used to facilitate this comparison.

In one embodiment, a mobilizing agent is administered to the subject if it is determined that the blood sample does not contain sufficient hematopoietic-reconstituting cells (i.e., with desired levels of one or more of AML-1, MYSM1, Hif1a, NPM-1, Profilin-1, phospho-GSK-3beta, SKP2, cbx7, Bmi-1, TCF1, Musashi-2, or FLI1). In another embodiment, the mobilizing agent is administered prior to assessing the level of the molecules in hematopoietic-reconstituting cells. In other embodiments, the process is iterative with assessment followed by mobilization and further assessments/mobilizations depending upon the results with the mobilizing agent.

The mobilizing agent may increase the number of hematopoietic-reconstituting cells from around 2×-2,000× or more. Ranges can be around 2×-10×, 10×-50×, 50×-100×, 100×-500×, 500×-1000×, 1000×-1500×, and 1500×-2000×.

In the case of inducers of one or more of AML-1, MYSM1, Hif1a, NPM-1, Profilin-1, phospho-GSK-3beta, SKP2, cbx7, Bmi-1, TCF1, Musashi-2, or FLI1, an increase of expression levels could be in the ranges of a 5% to greater than 100% increase. That includes, but is not limited to, about 5-10%; 10-20%; 20-30%; 30-40%; 40-50%; 50-60%; 60-70%; 70-80%; 80-90%; 90-100% or greater. For inhibitors the same ranges apply with a 0% low range.

Different agents may be used for mobilizing hematopoietic-reconstituting cells, depending on the types of blood cell and/or expression levels desired. In addition, the timing of the collection of the blood sample may affect the types of cells and/or expression levels of the cells collected. For example, it may be possible that expression of one or more of AML-1, MYSM1, Hif1a, NPM-1, Profilin-1, phosphor-GSK-3beta, SKP2, cbx7, Bmi-1, TCF1, Musashi-2, or FLI1 early in a mobilization differs from that later in the mobilization.

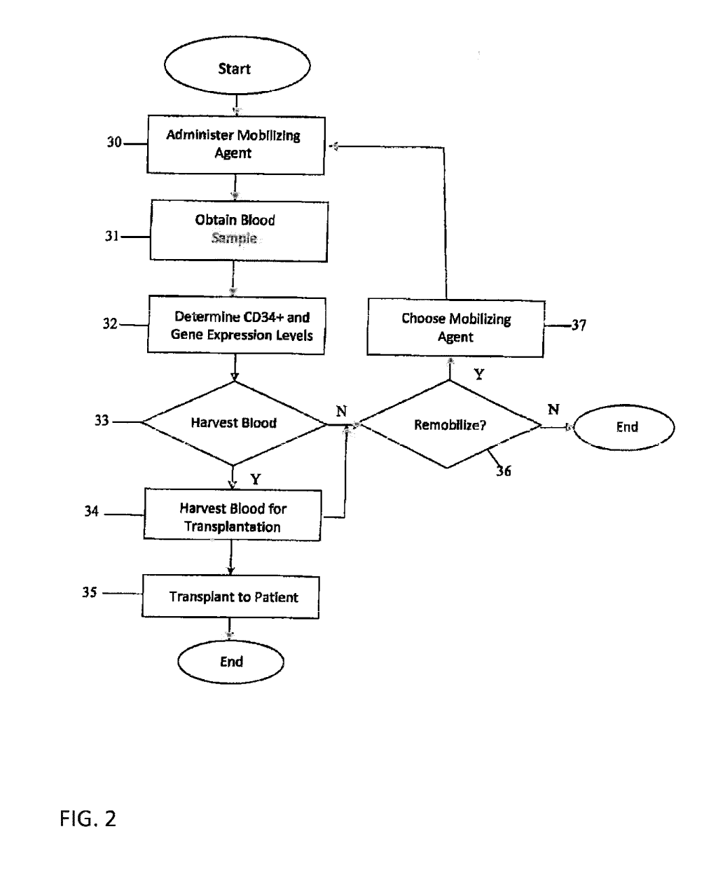

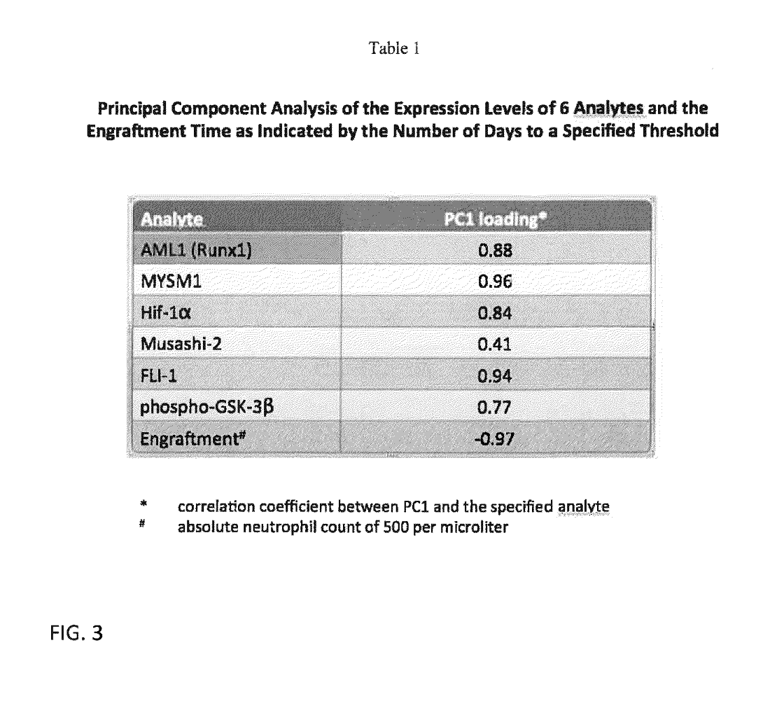

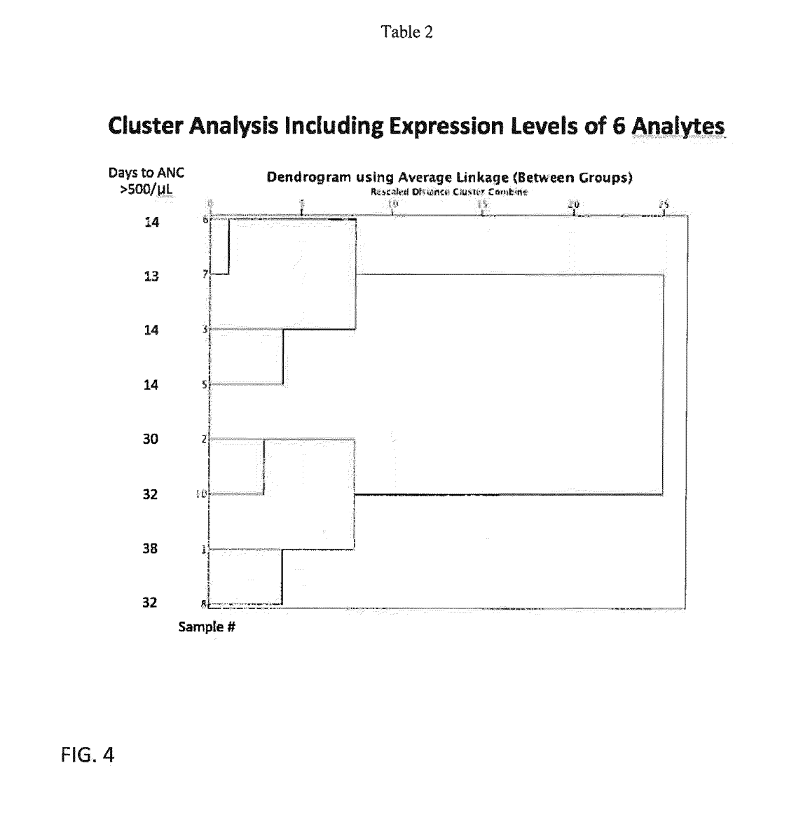

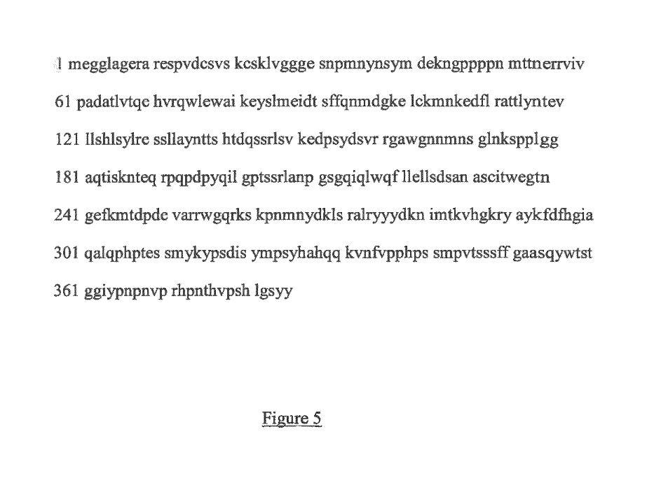

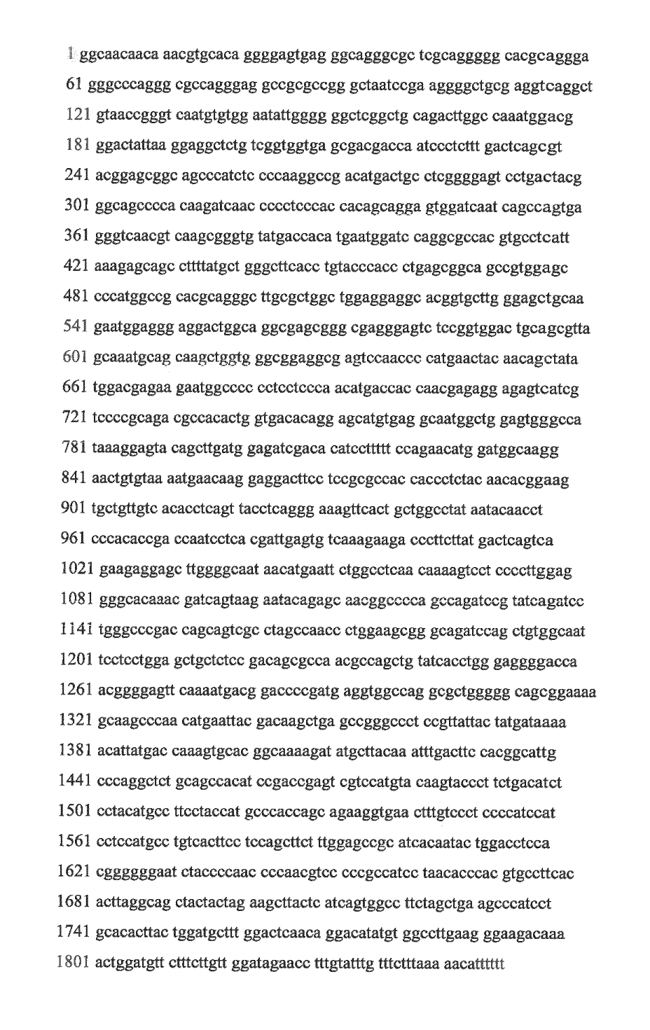

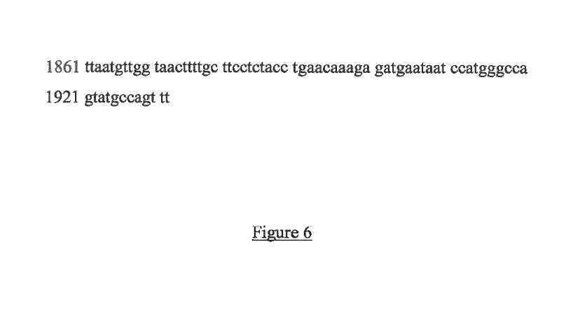

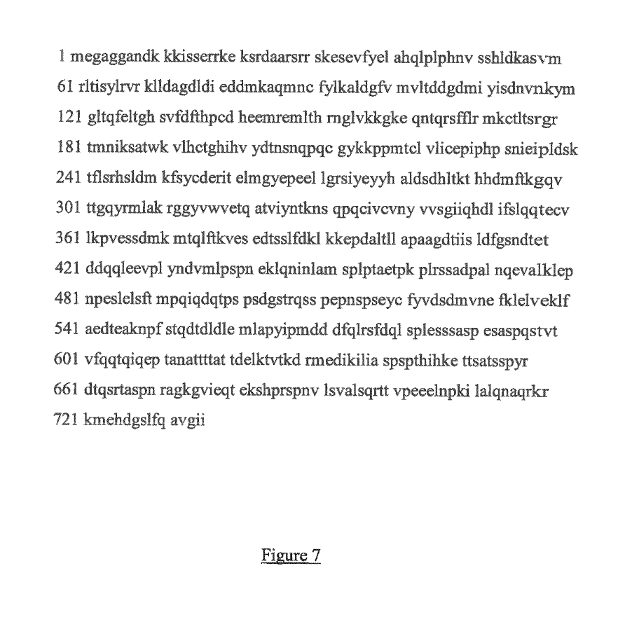

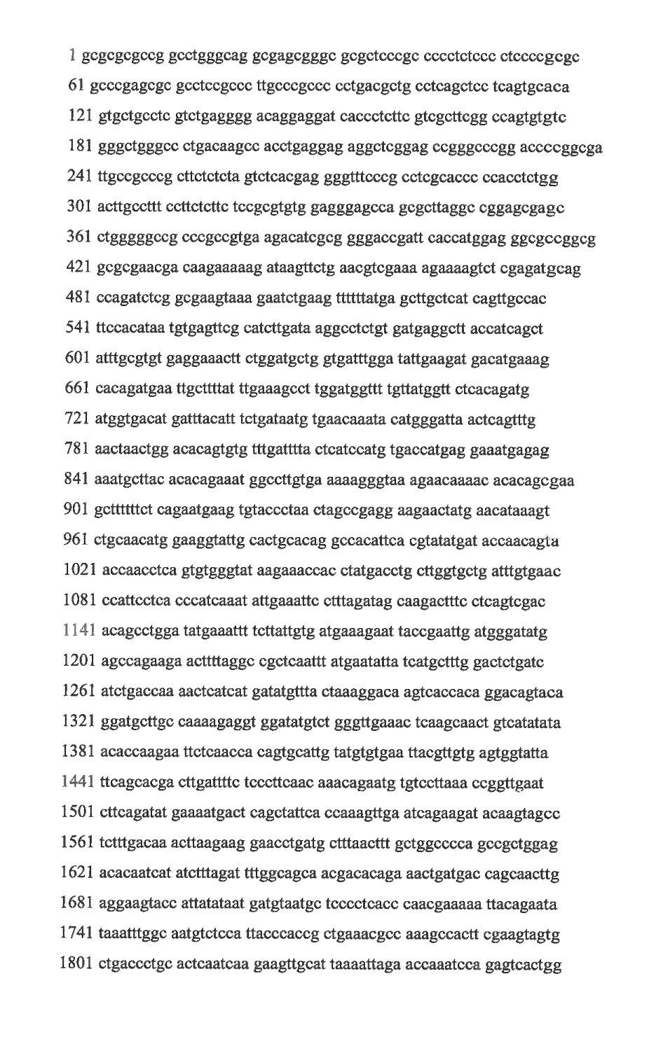

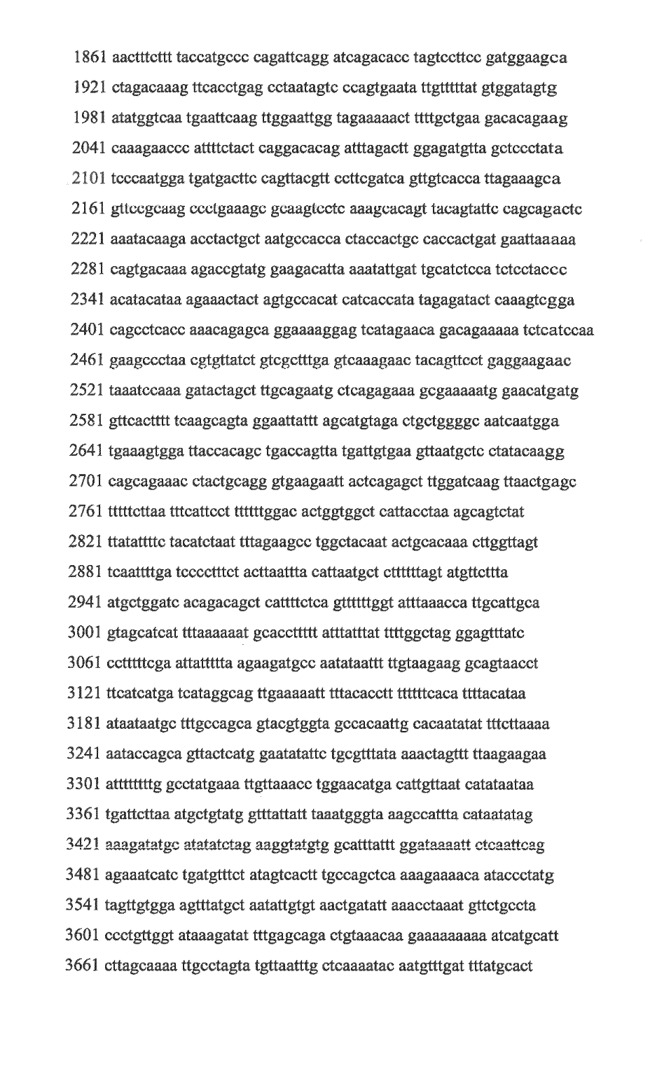

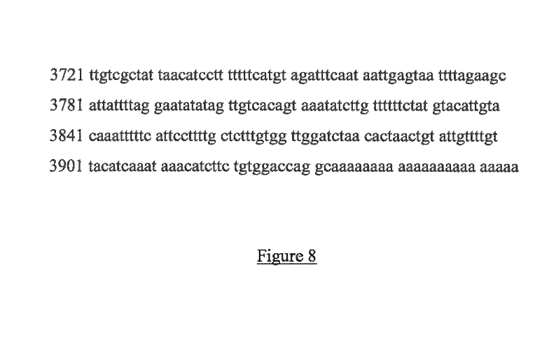

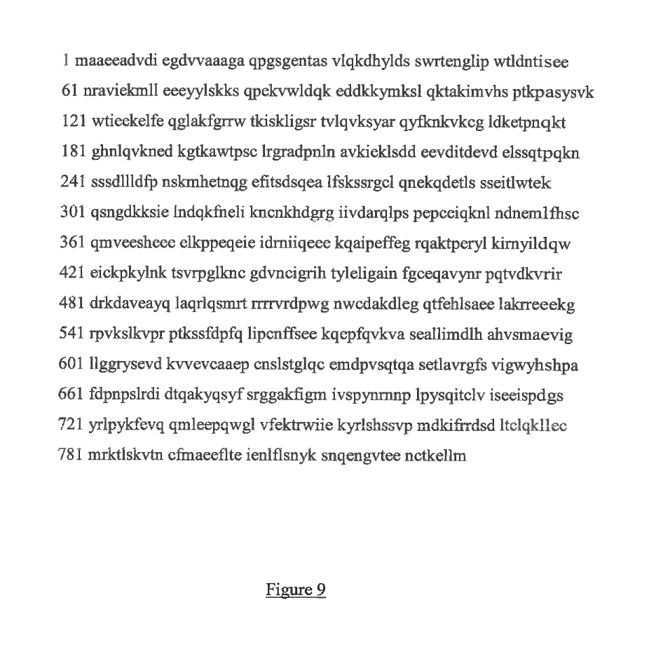

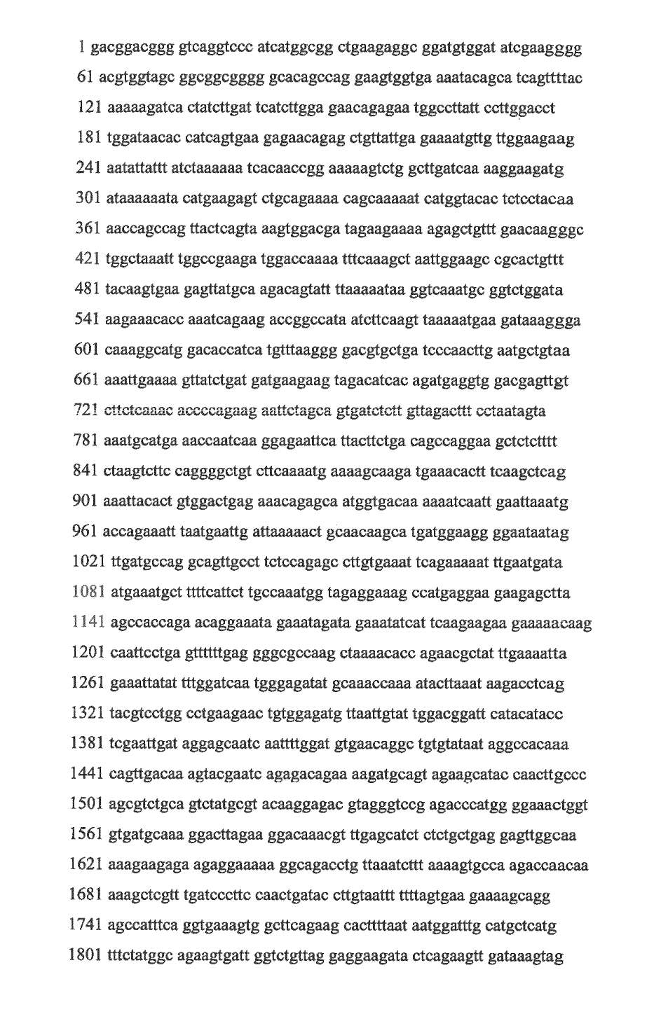

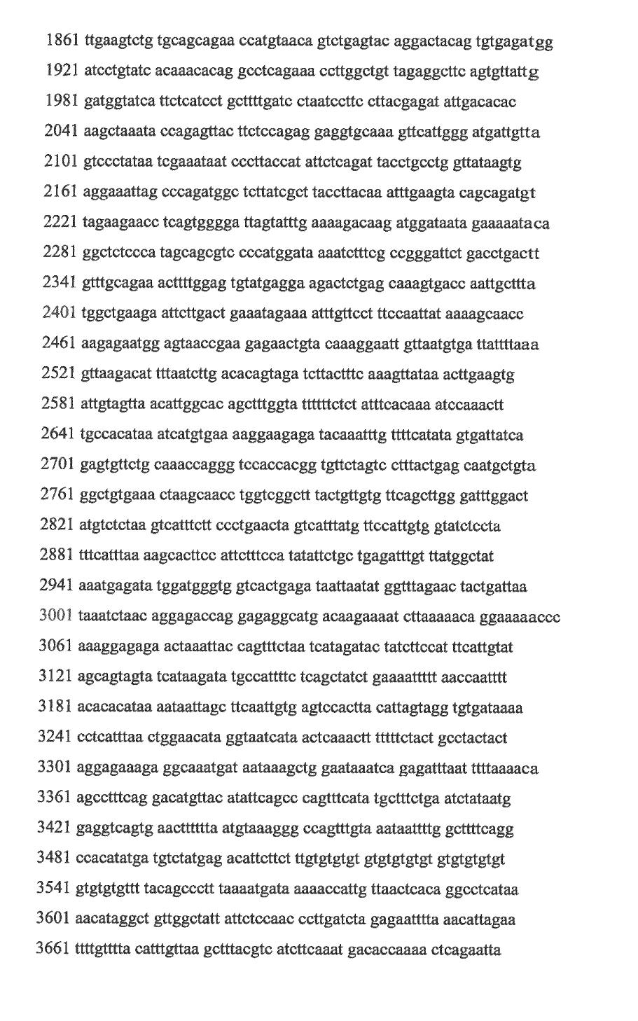

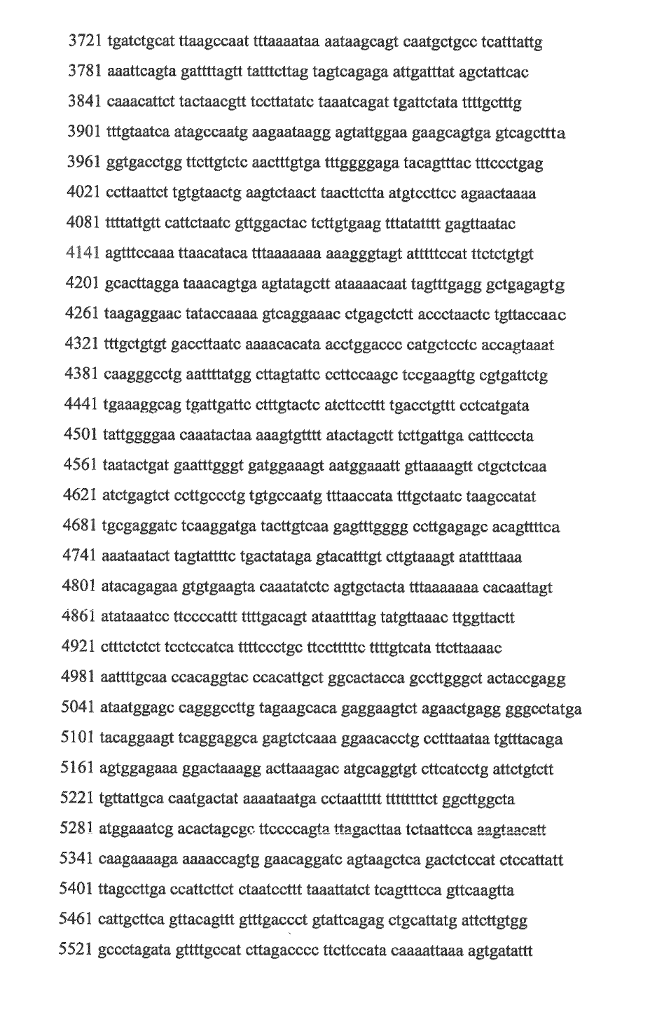

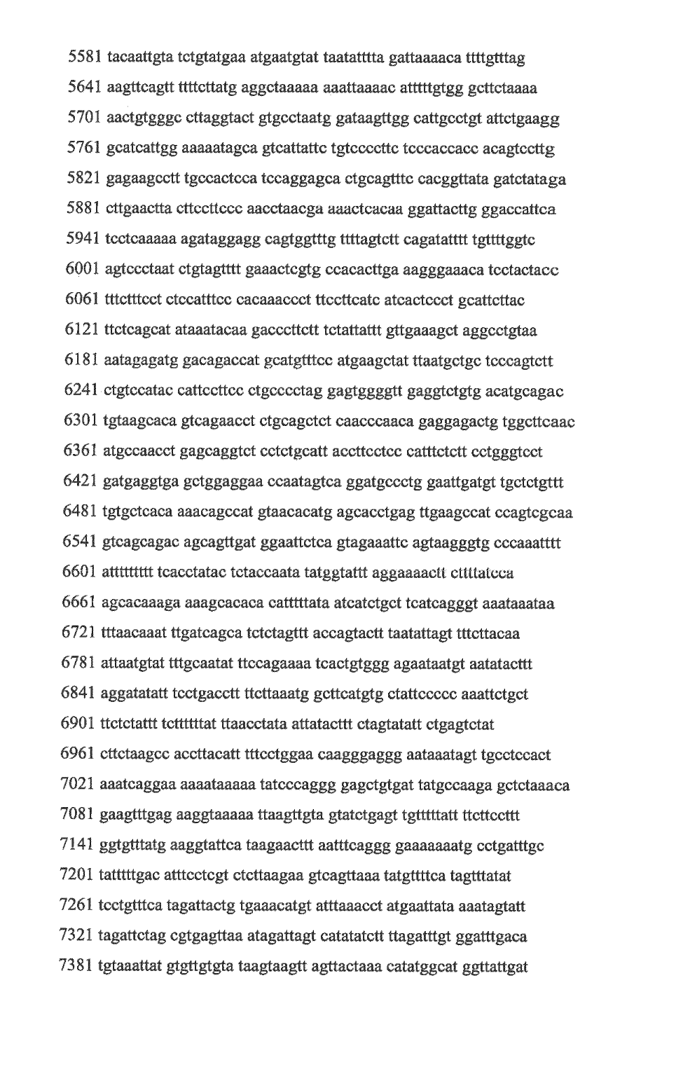

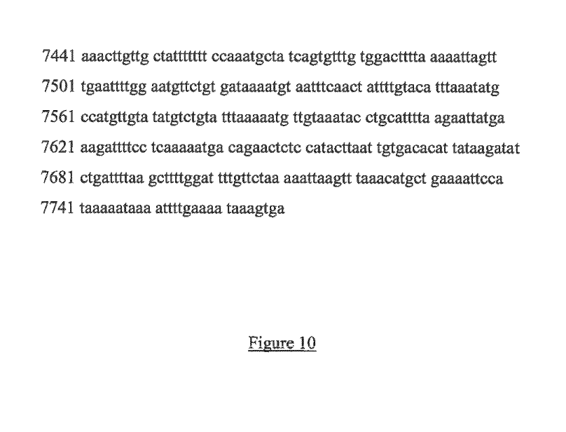

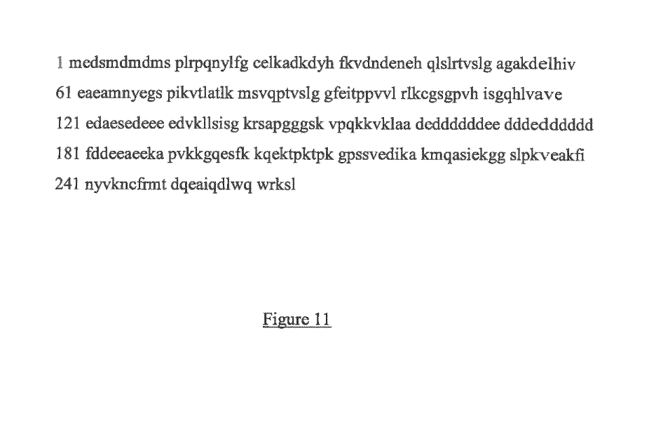

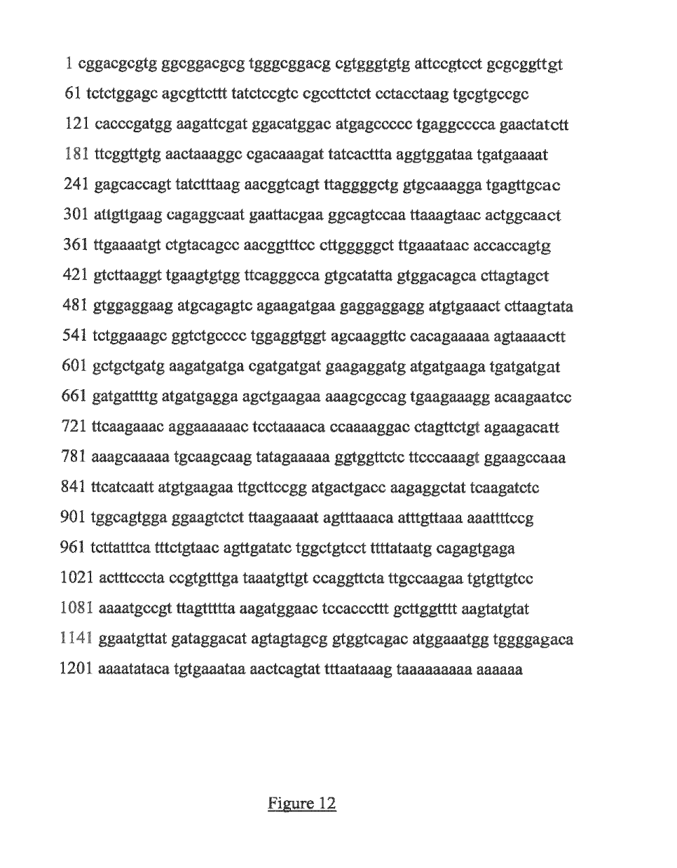

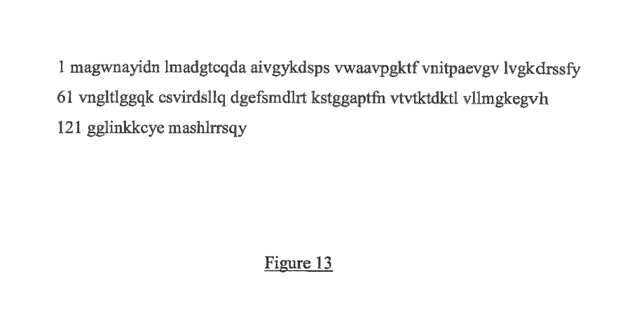

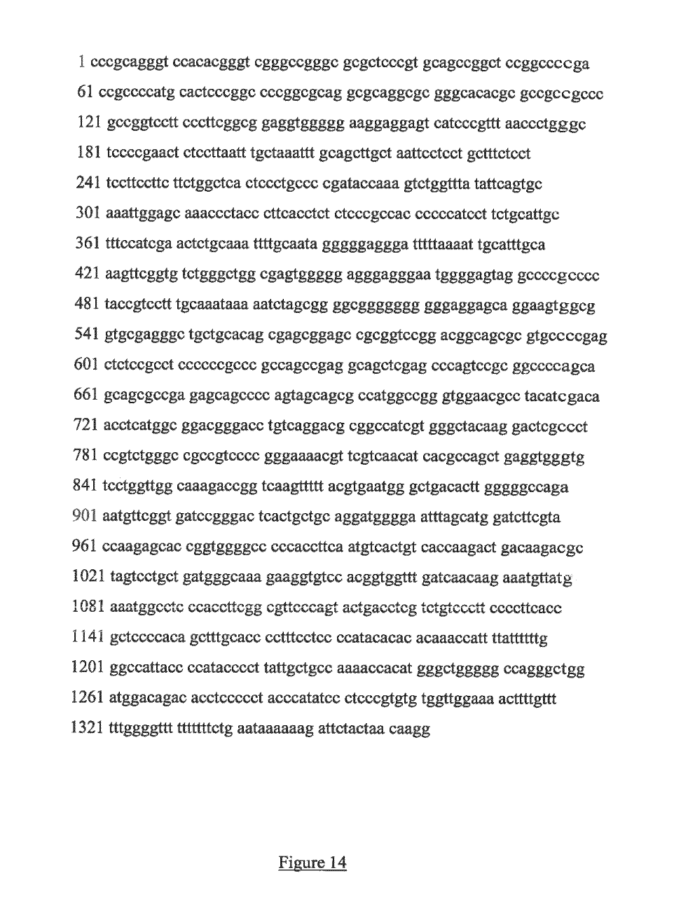

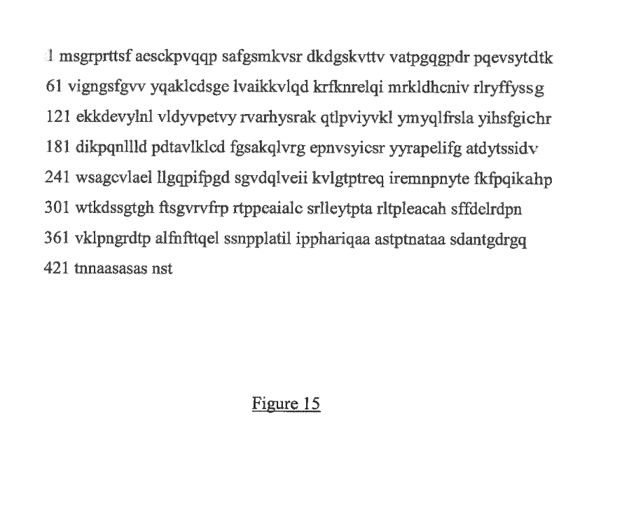

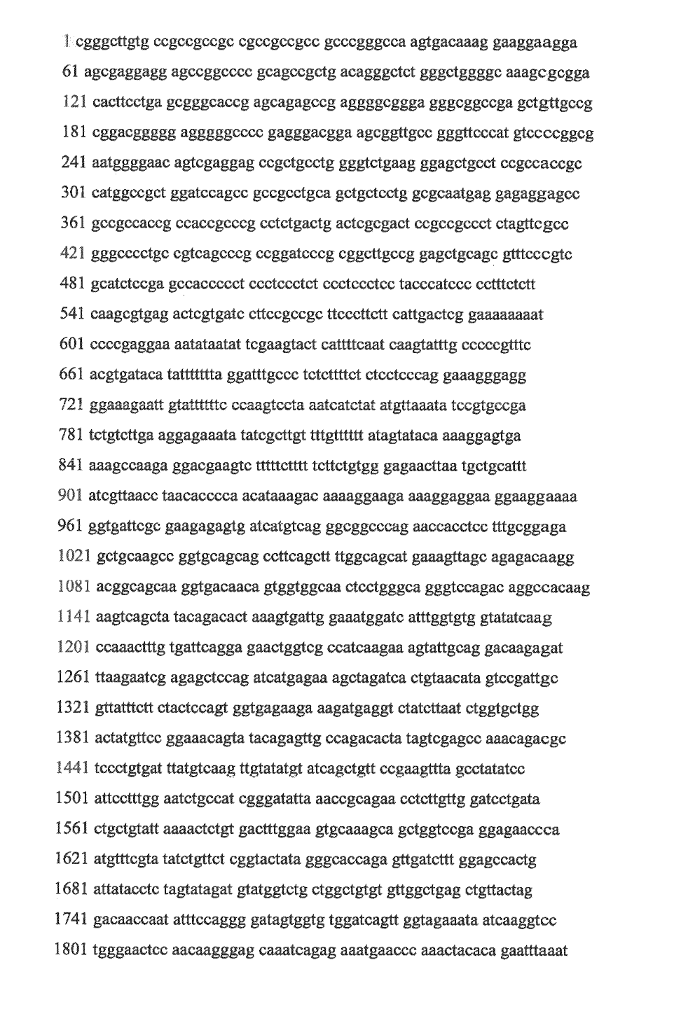

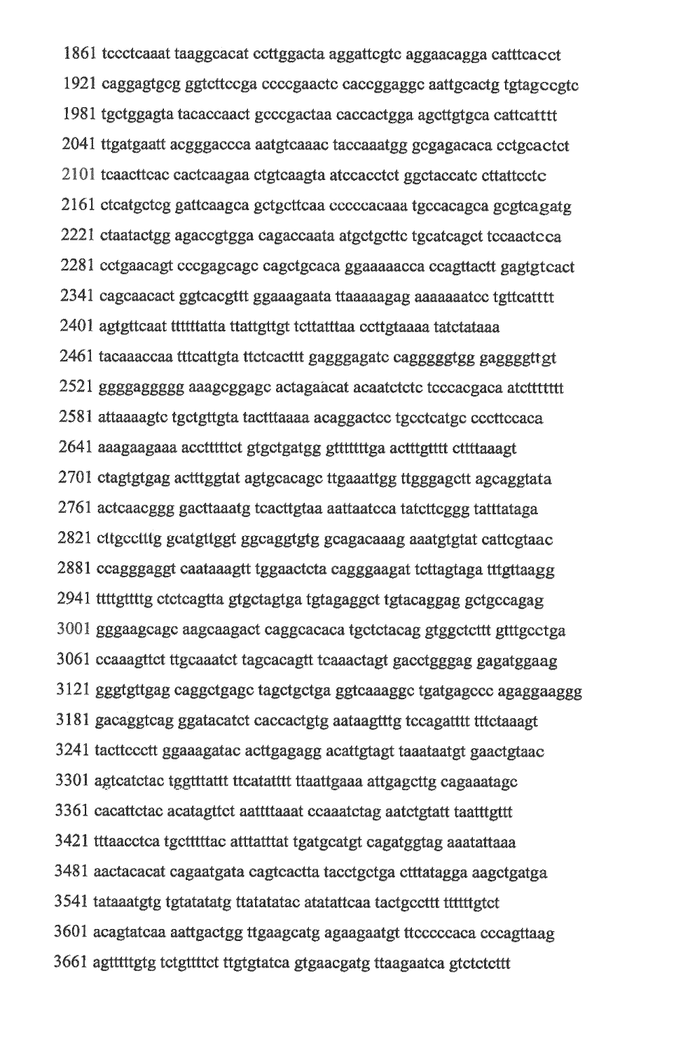

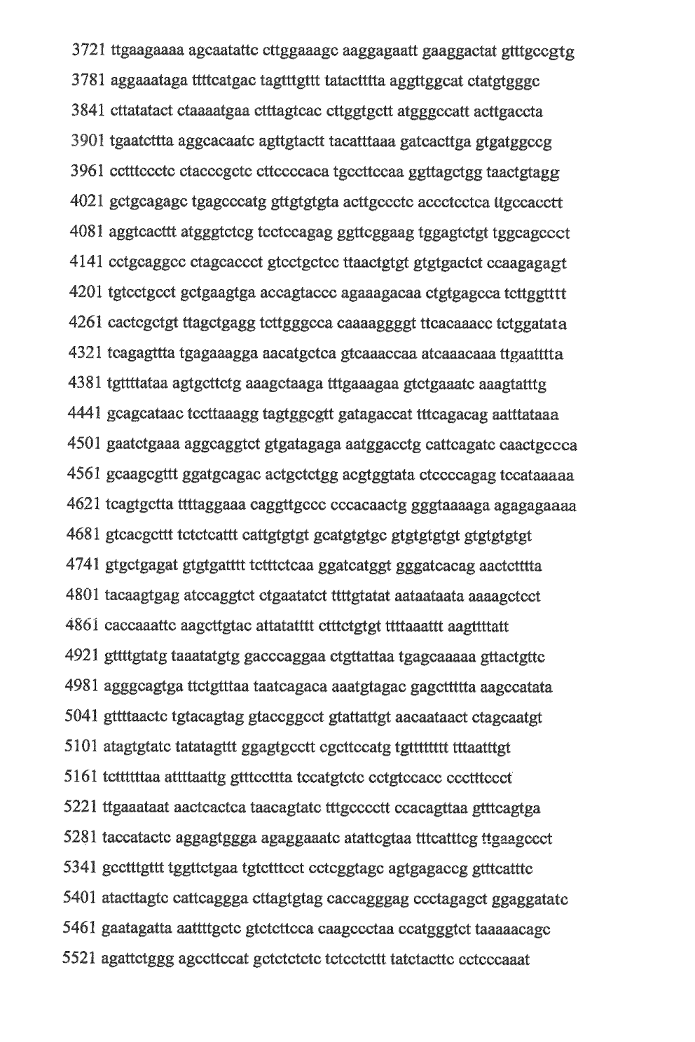

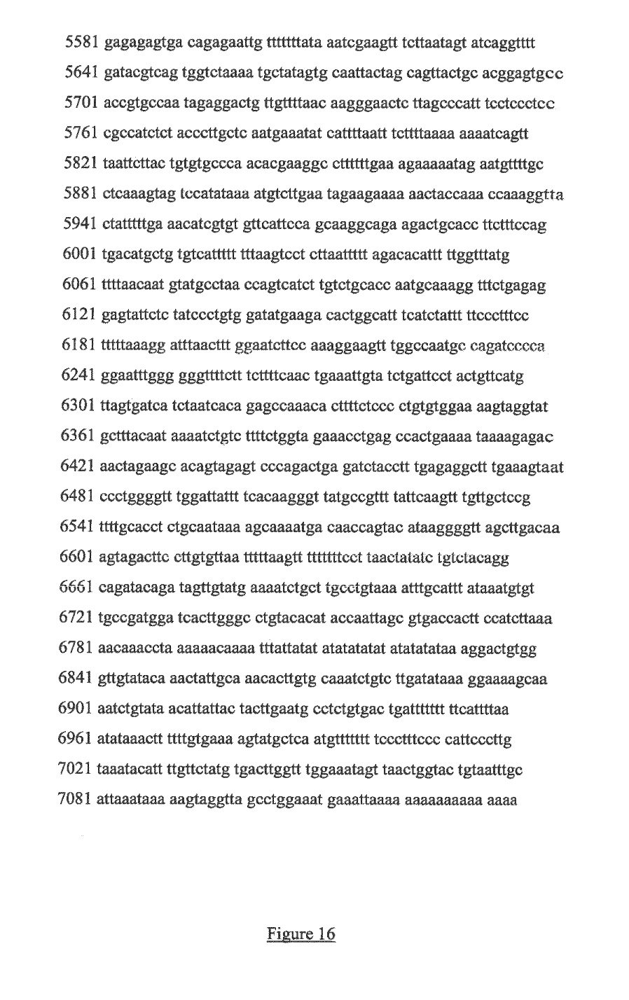

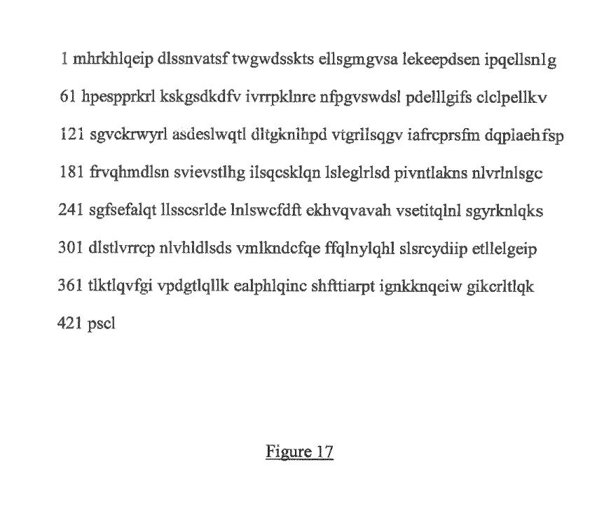

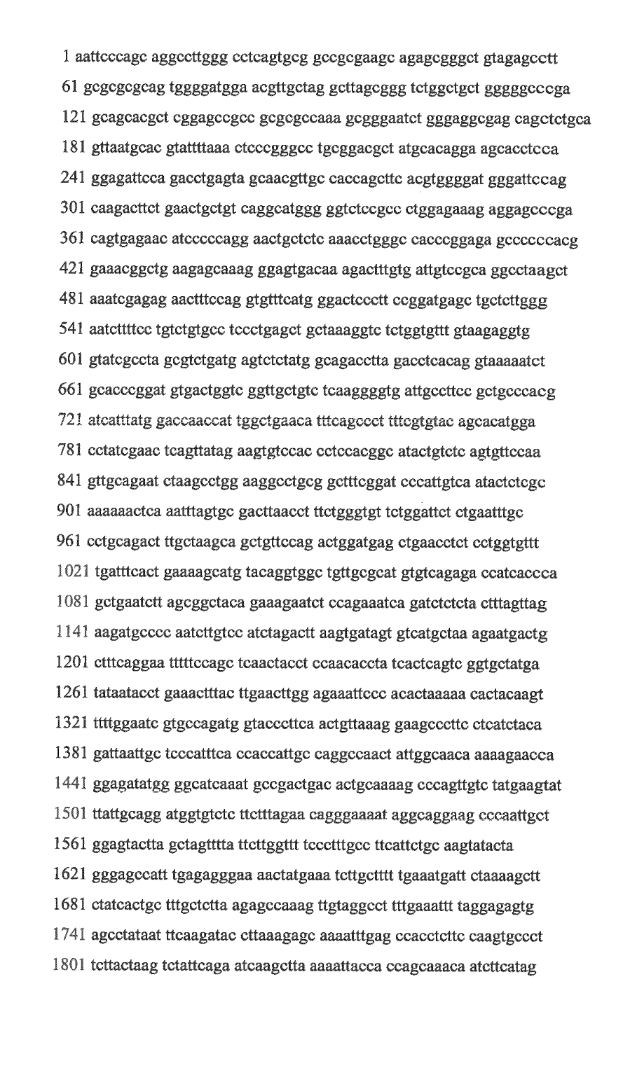

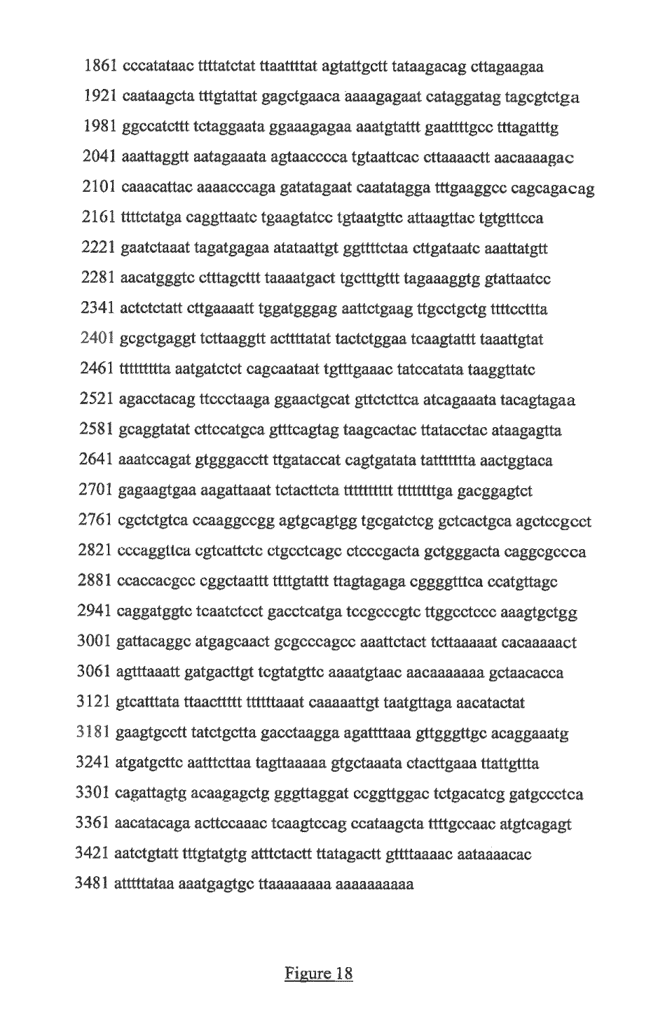

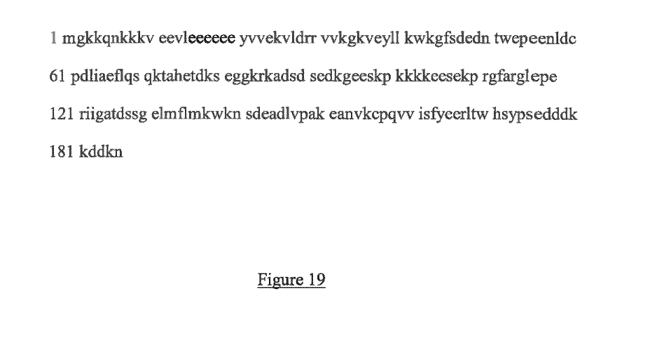

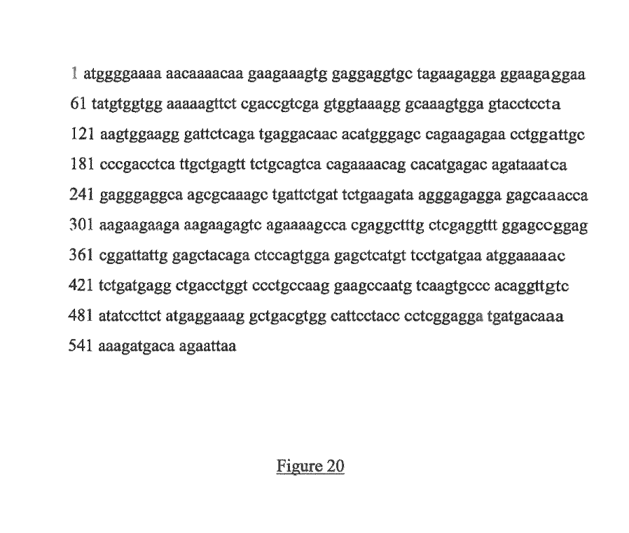

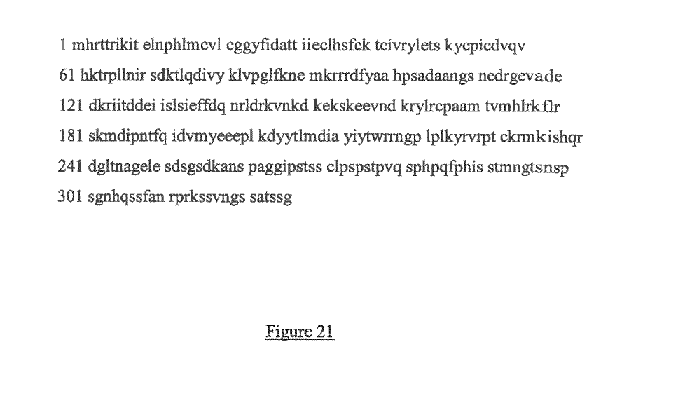

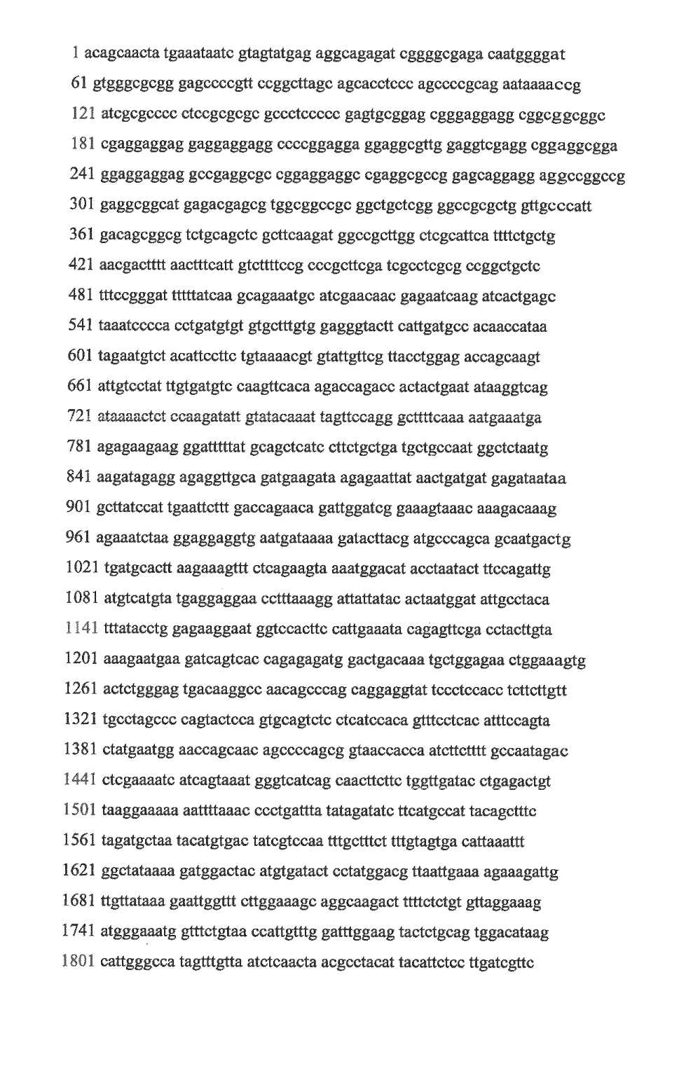

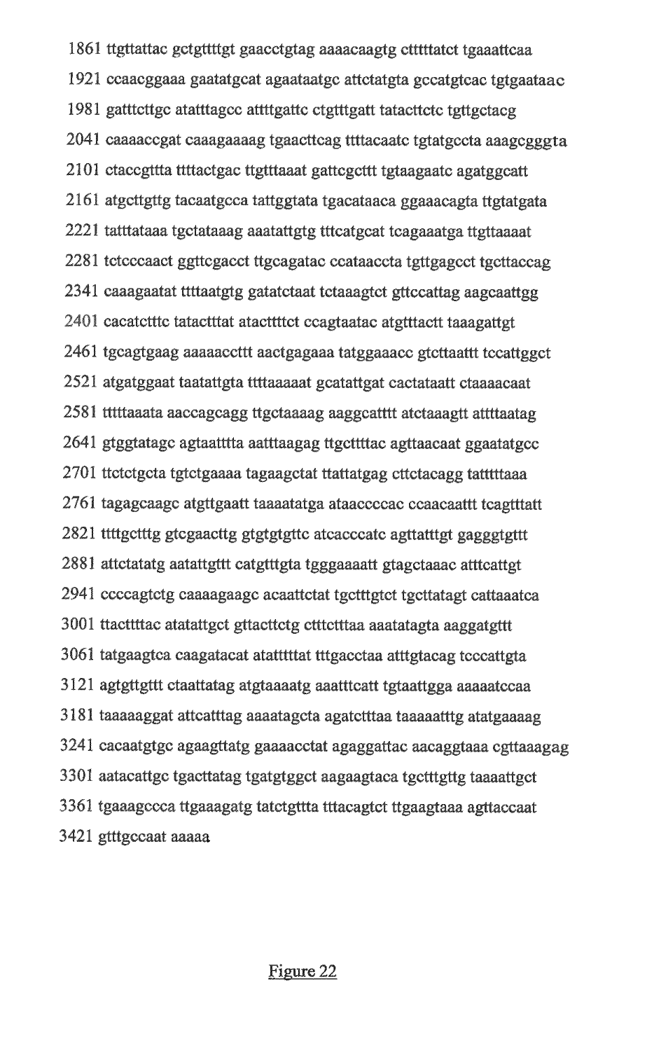

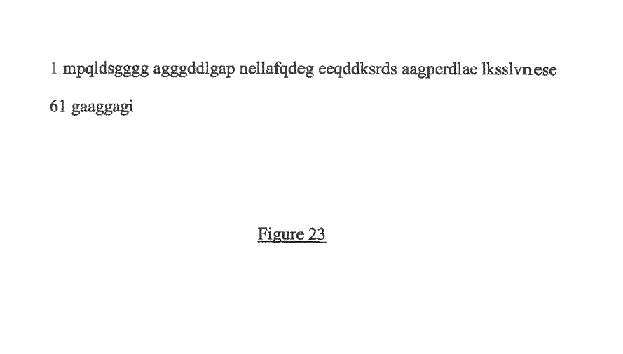

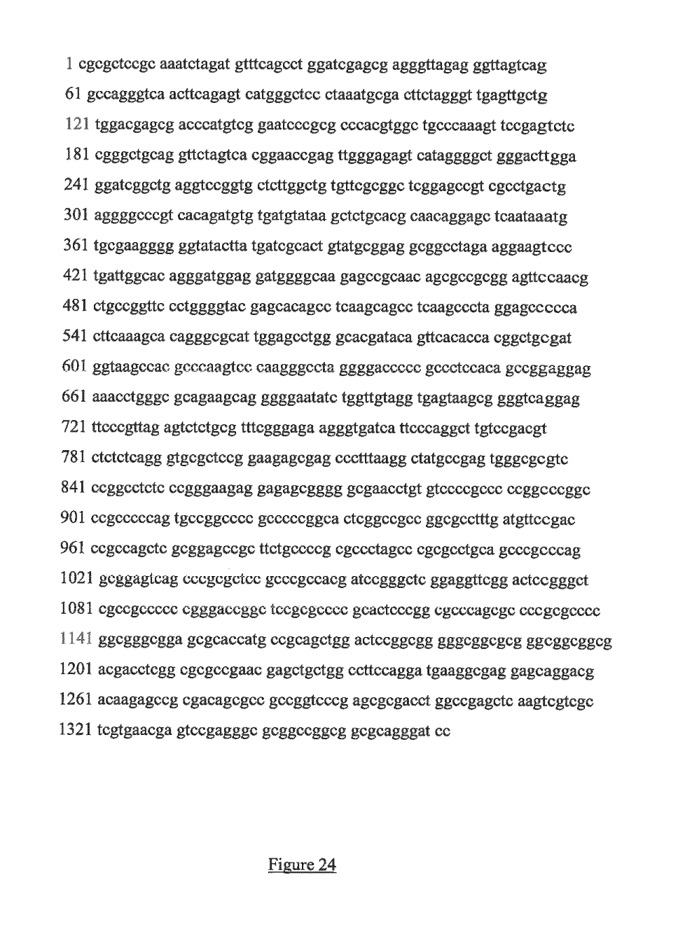

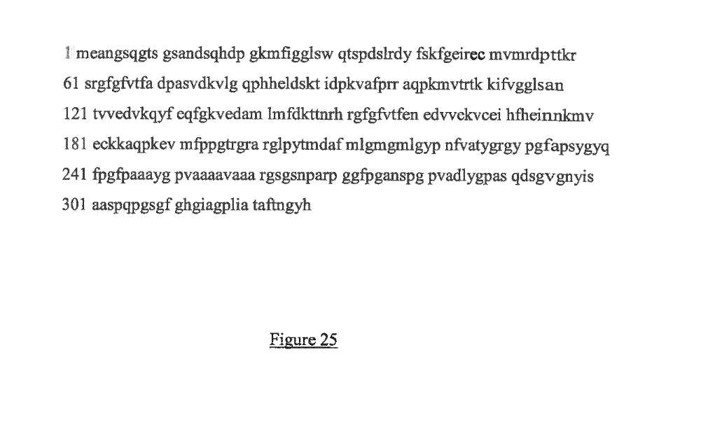

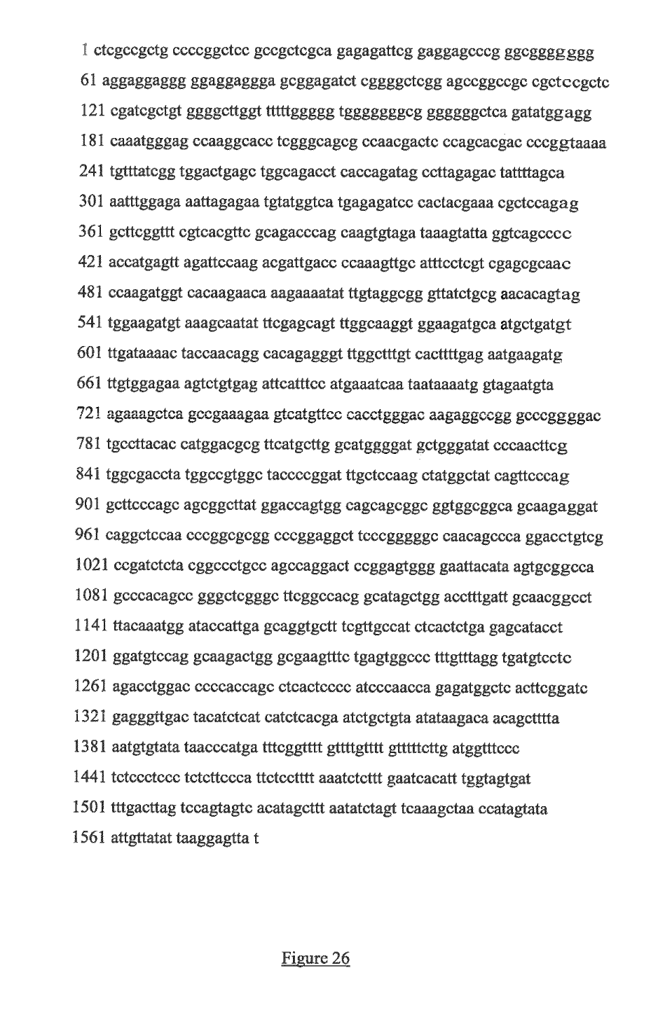

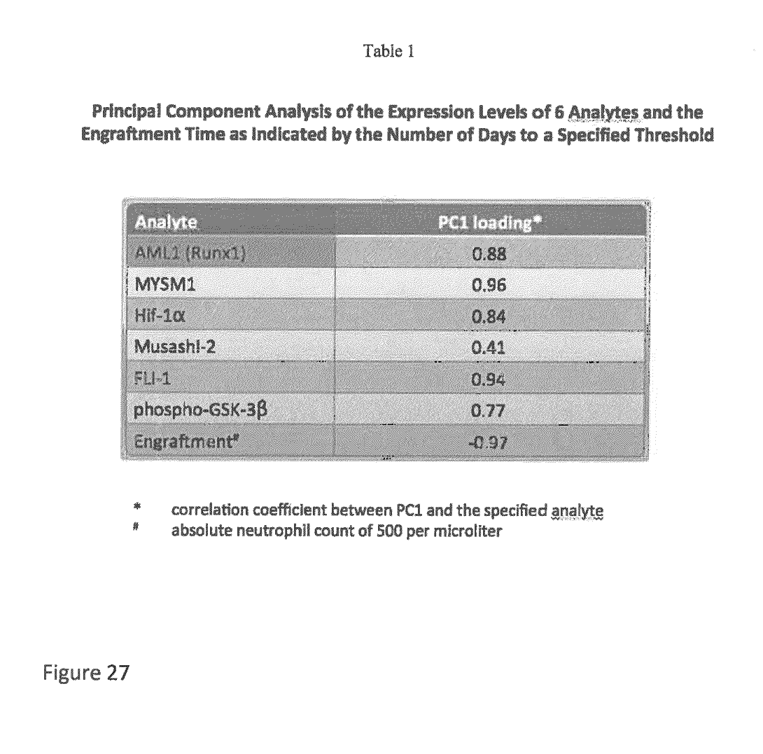

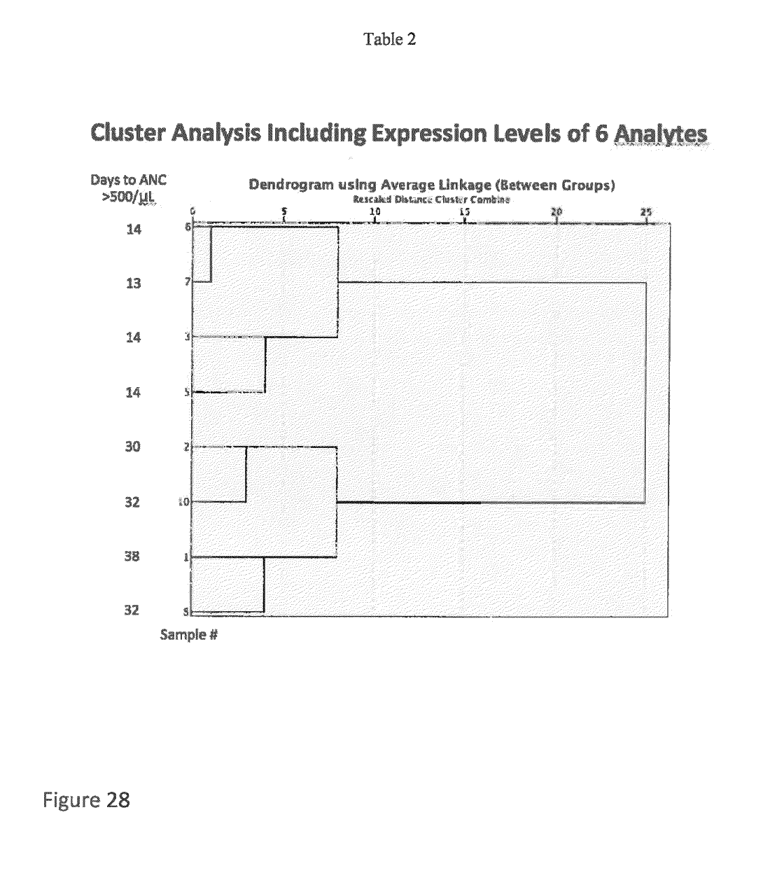

Referring to FIG. 2, a method for preparing a subject for donating blood for hematopoietic reconstitution in accordance with an embodiment of the present invention is illustrated in a flow chart. At step 30, a mobilizing agent is administered to the subject. A blood sample is obtained from the subject at step 31. At step 32, the co-expression levels of the blood sample are determined. A decision is made at step 33 whether the mobilized blood should be collected (i.e., harvested) from the subject based on the results obtained at step 32. If the decision is made to proceed with harvesting, the process continues to step 34 where the blood is collected before transplantation at step 35. The harvested blood may be stored prior to transplantation.

After harvesting the blood at step 33, a decision may be made at step 36 whether to remobilize the subject in order to obtain additional blood from the subject. If the decision is made to proceed with remobilizing the subject at step 36, the process proceeds to step 37 where the mobilizing agent is selected based on the desired characteristics of the additional blood to be drawn. The process then proceeds on to step 30. If the decision is made at step 36 not to remobilize the subject, the process ends.

The method illustrated in FIG. 2 may be modified such that one or more additional blood samples may be obtained from the subject after the initial mobilization has occurred at step 30. The subsequent samples may be obtained at various pre-determined intervals of time after mobilization has occurred because, as described above, the expression levels of the one or more of AML-1, MYSM1, Hif1a, NPM-1, Profilin-1, phospho-GSK-3beta, SKP2, cbx7, Bmi-1, TCF1, Musashi-2, or FLI1 in CD34⁺ cells collected may change in the time period following mobilization.

According to the methods of the present invention, CD34⁺ cells with the desired expression levels of one or more of AML-1, MYSM1, Hif1a, NPM-1, Profilin-1, phospho-GSK-3beta, SKP2, cbx7, Bmi-1, TCF1, Musashi-2, or FLI1 can be obtained from different mobilizations, and then administered to the patient in combination or sequentially.

In one embodiment the hematopoietic reconstituting cells that are administered to the subject are autologous. In another embodiment they are allogeneic.

In a further embodiment, the hematopoietic-reconstituting cells that are isolated from a subject for further administration are much more concentrated than they were in vivo. In fact these cells may form a substantially homogeneous population. Accordingly, the CD34⁺ cells expressing the desired levels of one or more of AML-1, MYSM1, Hif1a, NPM-1, Profilin-1, phospho-GSK-3beta, SKP2, cbx7, Bmi-1, TCF1, Musashi-2, or FLI1 can be used to directly create a source of cells to be administered at a later date and stored without further manipulation. Alternatively, the cells may be cultured, for example, expanded prior to or after storage. Accordingly, one can create a master cell bank with these cells, aliquots of which can be thawed and used for later administration with or without further expansion.

Because the methods described herein allow the isolation and concentration of the cell types described herein, the invention is also directed to novel compositions containing these cells at various levels of purity that have not been obtained before. These include about 5-10%, 10-20%, 20-30%, 30-40%, 40-50%, 50-60%, 60-70%, 70-80%, 80-90%, and 90-100%.

While this application exemplifies and focuses on the identification of a few specific molecules in CD34⁺ cells, the increased or decreased expression of which is correlated with greater potency, this technology more generally can be applied to ascertain any molecule that could be used as a potency marker. Thus, the invention can be more generally applied in terms of identifying molecules whose increased or decreased (or modified) expression is correlated with greater potency. This embodiment could involve assessing expression levels (modification, etc.) of molecules, involved in HRC function, from bone marrow of healthy adults, from the peripheral blood of G-CSF-treated adults, and from umbilical cord blood, and then selecting molecules that show either increased or decreased expression (modification) that correlates with the potency of the sample. In this regard, various other molecules have been associated with HRC function. It would, therefore, be a logical extension to apply the method used in this application to any of those other known molecules (as well as molecules discovered in the future that are suspected of being involved with HRC function).

In a more general sense, the method would apply to any experimental paradigm in which greater potency for any biological function can be distinguished between two (or more) different types of biological samples. Expression of molecules that is correlated with the potency in a sample could be ascertained. Having established the correlation, samples could be assessed for potency/function in the future by the gene expression pattern of the molecule.

### Stem Cells

The present invention can be practiced, preferably, using stem cells of vertebrate species, such as humans, non-human primates, domestic animals, livestock, and other non-human mammals. These include, but are not limited to, those cells described below.

Embryonic Stem Cells

The most well studied stem cell is the embryonic stem cell (ESC) as it has unlimited self-renewal and multipotent differentiation potential. These cells are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

A number of transcription factors and exogenous cytokines have been identified that influence the potency status of embryonic stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sa114, a mammalian Spalt transcription factor, is an upstream regulator of Oct4, and is therefore important to maintain appropriate levels of Oct4 during early phases of embryology. When Sa114 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass, and is down-regulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernible epiblast.

Non-Embryonic Stem Cells

Stem cells have been identified in most tissues. Perhaps the best characterized is the hematopoietic stem cell (HSC). HSCs are mesoderm-derived cells that can be purified using cell surface markers and functional characteristics. They have been isolated from bone marrow, peripheral blood, cord blood, fetal liver, and yolk sac. They initiate hematopoiesis and generate multiple hematopoietic lineages. When transplanted into lethally-irradiated animals, they can repopulate the erythroid neutrophil-macrophage, megakaryocyte, and lymphoid hematopoietic cell pool. They can also be induced to undergo some self-renewal cell division. See, for example, U.S. Pat. Nos. 5,635,387; 5,460,964; 5,677,136; 5,750,397; 5,681,599; and 5,716,827. U.S. Pat. No. 5,192,553 reports methods for isolating human neonatal or fetal hematopoietic stem or progenitor cells. U.S. Pat. No. 5,716,827 reports human hematopoietic cells that are Thy-1⁺ progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells. Accordingly, HSCs and methods for isolating and expanding them are well-known in the art.

Another stem cell that is well-known in the art is the neural stem cell (NSC). These cells can proliferate in vivo and continuously regenerate at least some neuronal cells. When cultured ex vivo, neural stem cells can be induced to proliferate as well as differentiate into different types of neurons and glial cells. When transplanted into the brain, neural stem cells can engraft and generate neural and glial cells. See, for example, Gage F. H., Science, 287:1433-1438 (2000), Svendsen S. N. et al, Brain Pathology, 9:499-513 (1999), and Okabe S. et al., Mech Development, 59:89-102 (1996). U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,564,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain. Accordingly, neural stem cells and methods for making and expanding them are well-known in the art.

Another stem cell that has been studied extensively in the art is the mesenchymal stem cell (MSC). MSCs are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

Another example of an adult stem cell is adipose-derived adult stem cells (ADSCs) which have been isolated from fat, typically by liposuction followed by release of the ADSCs using collagenase. ADSCs are similar in many ways to MSCs derived from bone marrow, except that it is possible to isolate many more cells from fat. These cells have been reported to differentiate into bone, fat, muscle, cartilage, and neurons. A method of isolation has been described in U.S. 2005/0153442.

Other stem cells that are known in the art include gastrointestinal stem cells, epidermal stem cells, and hepatic stem cells, which have also been termed “oval cells” (Potten, C., et al., Trans R Soc Lond B Biol Sci, 353:821-830 (1998), Watt, F., Trans R Soc Lond B Biol Sci, 353:831 (1997); Alison et al., Hepatology, 29:678-683 (1998).

Other non-embryonic cells reported to be capable of differentiating into cell types of more than one embryonic germ layer include, but are not limited to, cells from umbilical cord blood (see U.S. Publication No. 2002/0164794), placenta (see U.S. Publication No. 2003/0181269, umbilical cord matrix (Mitchell, K. E. et al., Stem Cells, 21:50-60 (2003)), small embryonic-like stem cells (Kucia, M. et al., J Physiol Pharmacol, 57 Suppl 5:5-18 (2006)), amniotic fluid stem cells (Atala, A., J Tissue Regen Med, 1:83-96 (2007)), skin-derived precursors (Toma et al., Nat Cell Biol, 3:778-784 (2001)), and bone marrow (see U.S. Publication Nos. 2003/0059414 and 2006/0147246), each of which is incorporated by reference for teaching these cells.

Strategies of Reprogramming Somatic Cells

Several different strategies such as nuclear transplantation, cellular fusion, and culture induced reprogramming have been employed to induce the conversion of differentiated cells into an embryonic state. Nuclear transfer involves the injection of a somatic nucleus into an enucleated oocyte, which, upon transfer into a surrogate mother, can give rise to a clone (“reproductive cloning”), or, upon explantation in culture, can give rise to genetically matched embryonic stem (ES) cells (“somatic cell nuclear transfer,” SCNT). Cell fusion of somatic cells with ES cells results in the generation of hybrids that show all features of pluripotent ES cells. Explantation of somatic cells in culture selects for immortal cell lines that may be pluripotent or multipotent. At present, spermatogonial stem cells are the only source of pluripotent cells that can be derived from postnatal animals. Transduction of somatic cells with defined factors can initiate reprogramming to a pluripotent state. These experimental approaches have been extensively reviewed (Hochedlinger and Jaenisch, Nature, 441:1061-1067 (2006) and Yamanaka, S., Cell Stem Cell, 1:39-49 (2007)).

Nuclear Transfer

Nuclear transplantation (NT), also referred to as somatic cell nuclear transfer (SCNT), denotes the introduction of a nucleus from a donor somatic cell into an enucleated ogocyte to generate a cloned animal such as Dolly the sheep (Wilmut et al., Nature, 385:810-813 (1997). The generation of live animals by NT demonstrated that the epigenetic state of somatic cells, including that of terminally differentiated cells, while stable, is not irreversible fixed but can be reprogrammed to an embryonic state that is capable of directing development of a new organism. In addition to providing an exciting experimental approach for elucidating the basic epigenetic mechanisms involved in embryonic development and disease, nuclear cloning technology is of potential interest for patient-specific transplantation medicine.

Fusion of Somatic Cells and Embryonic Stem Cells

Epigenetic reprogramming of somatic nuclei to an undifferentiated state has been demonstrated in murine hybrids produced by fusion of embryonic cells with somatic cells. Hybrids between various somatic cells and embryonic carcinoma cells (Solter, D., Nat Rev Genet, 7:319-327 (2006), embryonic germ (EG), or ES cells (Zwaka and Thomson, Development, 132:227-233 (2005)) share many features with the parental embryonic cells, indicating that the pluripotent phenotype is dominant in such fusion products. As with mouse (Tada et al., Curr Biol, 11:1553-1558 (2001)), human ES cells have the potential to reprogram somatic nuclei after fusion (Cowan et al., Science, 309:1369-1373(2005)); Yu et al., Science, 318:1917-1920 (2006)). Activation of silent pluripotency markers such as Oct4 or reactivation of the inactive somatic X chromosome provided molecular evidence for reprogramming of the somatic genome in the hybrid cells. It has been suggested that DNA replication is essential for the activation of pluripotency markers, which is first observed 2 days after fusion (Do and Scholer, Stem Cells, 22:941-949 (2004)), and that forced overexpression of Nanog in ES cells promotes pluripotency when fused with neural stem cells (Silva et al., Nature, 441:997-1001 (2006)).

Culture-Induced Reprogramming

Pluripotent cells have been derived from embryonic sources such as blastomeres and the inner cell mass (ICM) of the blastocyst (ES cells), the epiblast (EpiSC cells), primordial germ cells (EG cells), and postnatal spermatogonial stem cells (“maGSCsm” “ES-like” cells). The following pluripotent cells, along with their donor cell/tissue is as follows: parthogenetic ES cells are derived from murine oocytes (Narasimha et al., Curr Biol, 7:881-884 (1997)); embryonic stem cells have been derived from blastomeres (Wakayama et al., Stem Cells, 25:986-993 (2007)); inner cell mass cells (source not applicable) (Eggan et al., Nature, 428:44-49 (2004)); embryonic germ and embryonal carcinoma cells have been derived from primordial germ cells (Matsui et al., Cell, 70:841-847 (1992)); GMCS, maSSC, and MASC have been derived from spermatogonial stem cells (Guan et al., Nature, 440:1199-1203 (2006); Kanatsu-Shinohara et al., Cell, 119:1001-1012 (2004); and Seandel et al., Nature, 449:346-350 (2007)); EpiSC cells are derived from epiblasts (Brons et al., Nature, 448:191-195 (2007); Tesar et al., Nature, 448:196-199(2007)); parthogenetic ES cells have been derived from human oocytes (Cibelli et al., Science, 295L819 (2002); Revazova et al., Cloning Stem Cells, 9:432-449 (2007)); human ES cells have been derived from human blastocysts (Thomson et al., Science, 282:1145-1147 (1998)); MAPC have been derived from bone marrow (Jiang et al., Nature, 418:41-49 (2002); Phinney and Prockop, Stem Cells, 25:2896-2902 (2007)); cord blood cells (derived from cord blood) (van de Ven et al., Exp Hematol, 35:1753-1765 (2007)); neurosphere derived cells derived from neural cell (Clarke et al., Science, 288:1660-1663 (2000)). Donor cells from the germ cell lineage such as PGCs or spermatogonial stem cells are known to be unipotent in vivo, but it has been shown that pluripotent ES-like cells (Kanatsu-Shinohara et al., Cell, 119:1001-1012 (2004) or maGSCs (Guan et al., Nature, 440:1199-1203 (2006), can be isolated after prolonged in vitro culture. While most of these pluripotent cell types were capable of in vitro differentiation and teratoma formation, only ES, EG, EC, and the spermatogonial stem cell-derived maGCSs or ES-like cells were pluripotent by more stringent criteria, as they were able to form postnatal chimeras and contribute to the germline. Recently, multipotent adult spermatogonial stem cells (MASCs) were derived from testicular spermatogonial stem cells of adult mice, and these cells had an expression profile different from that of ES cells (Seandel et al., Nature, 449:346-350 (2007)) but similar to EpiSC cells, which were derived from the epiblast of postimplantation mouse embryos (Brons et al., Nature, 448:191-195 (2007); Tesar et al., Nature, 448:196-199 (2007)).

Reprogramming by Defined Transcription Factors

Takahashi and Yamanaka have reported reprogramming somatic cells back to an ES-like state (Takahashi and Yamanaka, Cell, 126:663-676 (2006)). They successfully reprogrammed mouse embryonic fibroblasts (MEFs) and adult fibroblasts to pluripotent ES-like cells after viral-mediated transduction of the four transcription factors Oct4, Sox2, c-myc, and Klf4 followed by selection for activation of the Oct4 target gene Fbx15 (FIG. 2A). Cells that had activated Fbx15 were coined iPS (induced pluripotent stem) cells and were shown to be pluripotent by their ability to form teratomas, although they were unable to generate live chimeras. This pluripotent state was dependent on the continuous viral expression of the transduced Oct4 and Sox2 genes, whereas the endogenous Oct4 and Nanog genes were either not expressed or were expressed at a lower level than in ES cells, and their respective promoters were found to be largely methylated. This is consistent with the conclusion that the Fbx15-iPS cells did not correspond to ES cells but may have represented an incomplete state of reprogramming. While genetic experiments had established that Oct4 and Sox2 are essential for pluripotency (Chambers and Smith, Oncogene, 23:7150-7160 (2004); Ivanona et al., Nature, 442:5330538 (2006); Masui et al., Nat Cell Biol, 9:625-635 (2007)), the role of the two oncogenes c-myc and Klf4 in reprogramming is less clear. Some of these oncogenes may, in fact, be dispensable for reprogramming, as both mouse and human iPS cells have been obtained in the absence of c-myc transduction, although with low efficiency (Nakagawa et al., Nat Biotechnol, 26:191-106 (2008); Werning et al., Nature, 448:318-324 (2008); Yu et al., Science, 318: 1917-1920 (2007)).

### MAPC

Human MAPCs are described in U.S. Pat. No. 7,015,037. MAPCs have been identified in other mammals. Murine MAPCs, for example, are also described in U.S. Pat. No. 7,015,037. Rat MAPCs are also described in U.S. Pat. No. 7,838,289.

These references are incorporated by reference for describing MAPCs first isolated by Catherine Verfaillie.

### Isolation and Growth of MAPCs

Methods of MAPC isolation are known in the art. See, for example, U.S. Pat. No. 7,015,037, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs have also been obtained my modified methods described in Breyer et al., Experimental Hematology, 34:1596-1601 (2006) and Subramanian et al., Cellular Programming and Reprogramming: Methods and Protocols; S. Ding (ed.), Methods in Molecular Biology, 636:55-78 (2010), incorporated by reference for these methods.

### Cell Culture

In general, cells useful for the invention can be maintained and expanded in culture medium that is available and well-known in the art. Also contemplated is supplementation of cell culture medium with mammalian sera. Additional supplements can also be used advantageously to supply the cells with the necessary trace elements for optimal growth and expansion. Hormones can also be advantageously used in cell culture. Lipids and lipid carriers can also be used to supplement cell culture media, depending on the type of cell and the fate of the differentiated cell. Also contemplated is the use of feeder cell layers.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007)).

Once established in culture, cells can be used fresh or frozen and stored as frozen stocks, using, for example, DMEM with 40% FCS and 10% DMSO. Other methods for preparing frozen stocks for cultured cells are also available to those of skill in the art.

### Pharmaceutical Formulations

In certain embodiments, the cell populations are present within a composition adapted for and suitable for delivery, i.e., physiologically compatible.

In some embodiments the purity of the cells for administration to a subject is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

Of course, samples found to have sufficient potency can be administered without any purification at all. But the inventor also envisions scenarios in which cells are created in vitro with the desired expression levels or possibly purified from in vivo and then expanded in vitro.

The choice of formulation for administering the cells for a given application will depend on a variety of factors. Prominent among these will be the species of subject, the nature of the condition being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

The dosage of the cells will vary within wide limits and will be fitted to the individual requirements in each particular case. In general, in the case of parenteral administration, it is customary to administer from about 0.01 to about 20 million cells/kg of recipient body weight. The number of cells will vary depending on the weight and condition of the recipient, the number or frequency of administrations, and other variables known to those of skill in the art. The cells can be administered by a route that is suitable for the tissue or organ. For example, they can be administered systemically, i.e., parenterally, by intravenous administration, or can be targeted to a particular tissue or organ; they can be administrated via subcutaneous administration or by administration into specific desired tissues.

The cells can be suspended in an appropriate excipient in a concentration from about 0.01 to about 5×10⁶ cells/ml. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

### Doing

Doses for humans or other mammals can be determined without undue experimentation by the skilled artisan, from this disclosure, the documents cited herein, and the knowledge in the art. The dose of cells/medium appropriate to be used in accordance with various embodiments of the invention will depend on numerous factors. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration.

The optimal dose of cells could be in the range of doses used for autologous, mononuclear bone marrow transplantation. For fairly pure preparations of cells, optimal doses in various embodiments will range from 10⁴ to 10⁸ cells/kg of recipient mass per administration. In some embodiments the optimal dose per administration will be between 10⁵ to 10⁷ cells/kg. In many embodiments the optimal dose per administration will be 5×10⁵ to 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

As an example, cell doses for umbilical cord blood or peripheral blood hematopoietic stem cell transplantation are somewhat different than bone marrow transplantation.

In various embodiments, cells may be administered in an initial dose, and thereafter maintained by further administration. Cells/medium may be administered by one method initially, and thereafter administered by the same method or one or more different methods. The levels can be maintained by the ongoing administration of the cells. Various embodiments administer the cells either initially or to maintain their level in the subject or both by intravenous injection. In a variety of embodiments, other forms of administration are used, dependent upon the patient's condition and other factors, discussed elsewhere herein.

Cells may be administered in many frequencies over a wide range of times. Generally lengths of treatment will be proportional to the length of the disease process, the effectiveness of the therapies being applied, and the condition and response of the subject being treated.

### Uses

Administering the cells is useful in any number of pathologies, including, but not limited to, those listed herein.

In addition, other uses are provided by knowledge of the biological mechanisms described in this application. One of these includes drug discovery. This aspect involves screening one or more compounds for the ability to affect the cell's ability to achieve any of the effects described in this application. Accordingly, the assay may be designed to be conducted in vivo or in vitro.

Gene expression can be assessed by directly assaying protein or RNA. This can be done through any of the well-known techniques available in the art, such as by FACS and other antibody-based detection methods, such as immunoassays (e.g., ELISA or Western blot) and PCR and other hybridization-based detection methods. Indirect assays may also be used for expression, such as the effect of gene expression.

A further use for the invention is the establishment of cell banks to provide cells for clinical administration. Generally, a fundamental part of this procedure is to provide cells that have a desired potency for administration in various therapeutic clinical settings.

In a specific embodiment of the invention, the cells are selected for having a desired potency for hematopoietic reconstitution (or the self-renewal and/or differentiation components).

Any of the same assays useful for drug discovery could also be applied to selecting cells for the bank as well as from the bank for administration.

Accordingly, in a banking procedure, the cells would be assayed for the ability to achieve any of the above effects. Then, cells would be selected that have a desired potency for any of the desired effects, and these cells would form the basis for creating a cell bank.

It is also contemplated that potency can be increased by treatment with an exogenous compound, such as a compound discovered through screening the cells with large combinatorial libraries. These compound libraries may be libraries of agents that include, but are not limited to, small organic molecules, antisense nucleic acids, siRNA DNA aptamers, peptides, antibodies, non-antibody proteins, cytokines, chemokines, and chemo-attractants. For example, cells may be exposed such agents at any time during the growth and manufacturing procedure. The only requirement is that there be sufficient numbers for the desired assay to be conducted to assess whether or not the agent increases potency. Such an agent, found during the general drug discovery process described above, could more advantageously be applied during the last passage prior to banking.

A further use is to assess the efficacy of the cell to achieve any of the above results as a pre-treatment diagnostic that precedes administering the cells to a subject. Moreover, dosage can depend upon the potency of the cells that are being administered. Accordingly, a pre-treatment diagnostic assay for potency can be useful to determine the dose of the cells initially administered to the patient and, possibly, further administered during treatment based on the real-time assessment of clinical effect.

It is also to be understood that the cells of the invention can be used not only for purposes of treatment, but also research purposes, both in vivo and in vitro to understand the mechanism involved normally and in disease models. In one embodiment, assays, in vivo or in vitro, can be done in the presence of agents known to be involved in the biological process. The effect of those agents can then be assessed. These types of assays could also be used to screen for agents that have an effect on the events that are promoted by the cells of the invention. Accordingly, in one embodiment, one could screen for agents in the disease model that reverse the negative effects and/or promote positive effects. Conversely, one could screen for agents that have negative effects in a non-disease model.

### Compositions

The invention is also directed to cell populations with specific potencies (i.e., desired expression levels) for achieving any of the effects described herein. As described above, these populations are established by selecting for CD34⁺ cells that have desired enhanced levels of one or more of AML-1, MYSM1, Hif1a, NPM-1, Profilin-1, phospho-GSK-3beta, SKP2, cbx7, Bmi-1, TCF1, Musashi-2, or FLI1. These populations are used to make other compositions, for example, a cell bank comprising populations with specific desired potencies and pharmaceutical compositions containing a cell population with a specific desired potency. Cultures can be established from in an in vivo source of the cells. Or cells can be created in vitro, such as by increasing the copy number of the genes or inducing/increasing endogenous gene expression.

Although the exemplified embodiment and the embodiment discussed in most detail in this application is directed to hematopoietic-reconstituting cells, the invention may also apply to other stem cells that can be used in transplantation. In other words, the issue involves the identification in those cells of molecules associated with a desired clinical outcome: embryonic stem cells, induced pluripotent cells, human progenitor cells, mesenchymal stem cells, mesenchymal stromal cells, human CD133+ stem cells, T lymphocytes, B lymphocytes, dendritic cells, regulatory T (Treg) cells, neural stem cells, neural progenitor cells, multipotent stem cells, pluripotent stem cells, endothelial progenitor cells, lymphocytes with chimeric antigen receptors, tumor infiltrating lymphocytes, genetically-engineered T lymphocytes, and natural killer cells. Accordingly, the following cells may be used for transplantation.

For predicting a clinical outcome after HRC transplantation by assessing expression levels of specific molecules in CD34⁺ cells in the transplanted inoculums, the clinical outcome can include, but is not limited to, relapse of the underlying cancer, secondary malignancy, graft-versus-host disease, bacterial infection, viral infection, fungal infection, failure to engraft, engraftment time, autoimmunity, myopathy, metabolic abnormalities, skeletal abnormalities, and dermatitis.

For mesenchymal stem cells, as the cells to be transplanted, the clinical outcome includes, but is not limited to, immunosuppression, altered autoimmune phenomena, immune tolerance, immune responsiveness, cartilage regeneration, range of motion, strength, articular joint function, pain, mobility, cognition, sight, inflammation, cardiac function, neurological function, and blood pressure.

For dendritic cells, as the cells to be transplanted, the clinical outcome includes, but is not limited to, immune responsiveness, immune tolerance, specific immunoglobulin levels, tumor regression, tumor size, overall survival, and progression-free survival.

For T lymphocytes, as the cells to be transplanted, the clinical outcome includes, but is not limited to, tumor regression, tumor size, overall survival, and progression-free survival.

For multipotent adult progenitor cells, as the cells to be transplanted, the clinical outcome includes, but is not limited to, immunosuppression, altered autoimmune phenomena, immune tolerance, immune responsiveness, cardiac function, neurological function, cognition, and blood pressure.

For neural stem cells/neural progenitor cells, as the cells to be transplanted, the clinical outcome includes, but is not limited to, neurological function, cognition, nerve conduction, and inflammation.

### Example 1

In the inventor's studies the clinical outcome designated “engraftment” refers to: the speed of engraftment as the variable that the inventor desires to maximize. It was assessed by the time in days it takes to reach the following milestones in the peripheral blood: >20,000 platelets per microliter; >50,000 platelets per microliter; >100,000 platelets per microliter; >500 neutrophils per microliter.

### Background

Umbilical cord blood (UCB) units are used as a source of hematopoietic stem cells (HSC) for transplantation as treatment for various malignant or non-malignant causes. Bone marrow or peripheral blood from individuals treated to mobilize HSC from the bone marrow into the peripheral blood are alternative sources of HSC for transplantation. The current selection criteria for use of UCB units for transplantation includes the total nucleated cell count. With this procedure, approximately 20% of recipients experience primary engraftment failure or prolonged engraftment time (Eapen M, et al, Lancet Oncol, 2010: 11:653-660; Kurtzberg J, et al, Blood, 2008: 112:4318-4327; Martin P L, et al, Biol Blood Marrow Transplant, 2006, 12:184-194; Barker J N, et al, Blood, 2010, 115:1843-1849). The deficiency in the UCB units results in part from inadequate potency of the HSC in the unit (Page K M, et al, Biol Blood Marrow Transplant, 2011, 17:1362-1374).

Using our high-resolution flow cytometric technology, we have studied HSC for measures of potency. Our conception has been that expression levels of molecules shown to be important for hematopoiesis in experimental studies may have utility in assessing the potency of HSC in terms of engraftment after transplantation. Our studies have demonstrated the capacity of our approach to find significant associations between molecular expression levels in a treatment sample and a specific clinical outcome.

Our data have demonstrated that expression levels of various molecules differ significantly in the CD34⁺ cells from sources of varying potencies (e.g. UCB v. PB). Many of the molecular expression levels demonstrated highly significant bivariate correlations. We found many bivariate correlations with r>0.8 (x). Thus, our technology has sufficient reproducibility, precision, and quantitative quality to reveal significant intermolecular relationships. This capability is important because it allows us to use powerful multivariate analytical tools, such as, principal component analysis, factor analysis, and cluster analysis to find meaning in our datasets. The capability of obtaining correlations of the expression of genes with correlation coefficients great than 0.6 is the subject of a U.S. patent application Ser. No. 13/829,557.

### Testing the Model: Pharmacological Enhancement of UCB HSC for Transplantation

A major deficiency of UCB is low HSC content. A strategy that has been developed to address this deficiency is a pulse incubation of UCB cells with a small molecule, Prostaglandin E2 (PGE2), that enhances the potency of the HSC in terms of engraftment in a clinical trial. (Goessling W, et al, Cell Stem Cell, 2011, 8:445-458).

In order to assess molecular expression level correlates of potency in UCB HSC, we treated UCB mononuclear cells with PGE2 and assessed the expression of phospho-GSK-33.

Using our technology and analytical methods we were able to detect differences in expression of phospho-GSK-3β in CD34⁺ cells from UCB treated with the reagent vehicle (DMSO) versus PGE2. This is consistent with our previous finding that phospho-GSK-3β is a potency biomarker.

### Testing the Model: Molecular Expression in UCB Used for Transplantation

We analyzed 23 samples of UCB units that had previously been used for transplantation. A collaborating clinician selected the samples based on clinical outcome: half with fast neutrophil engraftment and half with slow neutrophil engraftment. Engraftment time is, in a sense, an inverse measure: better values are lower. We found several bivarate correlation differences in the samples that were stratified by engraftment time, indicating that the molecular organization of the cells that gave fast engraftment is distinct from the molecular organization of the cells that gave slow engraftment.

We then used PCA and found that engraftment time varied inversely with levels of many molecules. These included AML1, MYSM1, Hif1α, phospho-Akt(thr308), FLI1, Mcl1, phospho-GSK-3β, Musashi-2, and NPM1.

PCA is a procedure to find vectors in n-dimensional space that account for the variance in the dataset with n variables. The first principal component (PC1) explains the greatest amount of the variance in the dataset. The loading is the correlation coefficient between the variable and the unseen principal component. In the case of engraftment time, we observed that it was inversely or negatively correlated to PC whereas many other analyte expression levels were directly or positive correlated to PC1. The value of the loadings that we obtained were significant (>0.4) which supports our contention that we have uncovered an important relationship. Finally, it should be noted that we used the covariance matrix in PCA but use of the correlation matrix gave similar results. Also, we employed varimax rotation but again substantially similar results were obtained without rotation.

Because we wanted to limit the number of analytes considered, we pared the number of analytes in PCA from 9 to 6. The results of PCA with 6 analytes follows:

Our goal was to classify samples based on molecular expression levels so that samples with subsequent fast engraftment can be segregated from samples with subsequent slow engraftment. The results of PCA indicate that engraftment time is associated with a variety of molecular expression levels. We used these same analytes to determine whether the samples segregated from each other based on engraftment time by performing hierarchical cluster analysis. The results follow:

The method used for this analysis was between groups linkage based on squared Euclidean distance. The horizontal length of the lines that connect the samples indicates the relatedness of the samples based on the expression levels of the 6 analytes. The sample numbers are given to the left of the plot and the corresponding engraftment time is shown as well. It should be noted that only 8 samples were included in the cluster analysis. Samples not assessed for all 6 molecules selected could not be analyzed.

The results indicate that the 4 samples associated with fast engraftment clustered together and the 4 samples associated with slow engraftment clustered together. Most saliently, the 2 clusters were separated by the maximal distance possible. These results indicate that molecular expression levels for AML1, MYSM1, Hif1α, Musashi-2, FLI1, and phospho-GSK-3β could be used in a blind fashion to segregate samples that resulted in distinct clinical outcomes upon subsequent transplantation.

Musashi-2 and phospho-GSK-3β had been originally identified as markers of potency. Also, as shown above, phospho-GSK-3β is a potency marker in the PGE2 investigation. The other analytes had not been previously identified.

Accordingly, the test models show that we have obtained our primary objective, which is, to use molecular expression levels of CD34⁺ cells from UCB units to predict subsequent clinical outcome in terms of engraftment time.

Accordingly, the invention involves exploiting the assays/analytic procedures in our earlier work (e.g., application Ser. No. 13/829,557), e.g., assay the ratio of one or more of the genes described to one or more of the other genes described. This allows the methods in this disclosure, which include predicting the potency of a given sample of CD34⁺ cells for transplantation as well as the other uses described (e.g., agents that affect potency).

### Testing the Model: Myeloablated Patients

In view of the above the invention is also directed to the method of measuring the expression levels of AML1 (also known as Runxl), MYSM1, Hif1α, and FLI1 in the CD34-expressing hematopoietic stem cells in order to assess the likelihood of early hematopoietic engraftment in myeloablated patients transplanted with the CD34-expressing hematopoietic stem cells.

In order to assess the differential capability of CD34⁺ cells from umbilical cord blood samples in terms of their ability to effect engraftment after myeloablation, we assessed the expression levels of 4 molecules in these cells from 19 samples that had previously been used to reconstitute hematopoiesis in patients. Of these 19 samples, 8 were associated with fast engraftment of neutrophils (12-16 days) and 11 were associated with slow engraftment of neutrophils (>25 days).

The 4 molecules analyzed were AML, MYSM1, Hif1α, and/or FLI1. AML is a transcription factor previously associated with the differentiation of hematopoietic stem cells into mature blood components. MYSM1 is a metalloprotease that deubiquitinates histone 2A, thereby cancelling transcriptional repression. Hif1α, is a transcription factor especially active in tissues with low oxygen concentrations such as the bone marrow. FLI1 is also a transcription factor.

In this study, AML was found to be significantly correlated with time to engraftment of neutrophils (r=−0.47; p=0.04). The expression levels of the other molecules assessed were not significantly associated with the time to engraftment; however, they were highly correlated with the expression level of AML1.

In multiple linear regression analysis, we found that the number of infused CD34⁺ cells and the expression level of AML1 in the CD34⁺ cells were significant, independent predictors of the time to neutrophil engraftment. The multiple linear regression analysis demonstrated that both the number of CD34+ cells (p=0.01) and the expression level of AML1 (p=0.05) independently predicted engraftment time in the patients.

These results demonstrate the use of molecular expression levels in transplanted cells to enhance the prediction of a desired clinical outcome.

### Testing the Model: Myeloablated Patients

We also measured the expression levels of phospho-GSK-3, MYSM1, and/or HoxB4 in order to assess the likelihood of early hematopoietic engraftment in myeloablated patients transplanted with the CD34-expressing hematopoietic stem cells.

In order to assess the differential capability of CD34⁺ cells from samples of MBC in terms of their ability to effect engraftment after myeloablation, we assessed the expression levels of 23 molecules in these cells from 24 MBC samples that had previously been used to reconstitute hematopoiesis in patients. Of these 24 samples, 13 were associated with fast engraftment of platelets (<50 days) and 11 were associated with slow engraftment of neutrophils (>50 days).

All of these molecules assessed were previously associated with the function of hematopoietic reconstitution.

Using linear discriminant analysis, we found that the expression levels of phospho-GSK-33, HoxB4, and MYSM1 along with the number of CD34+ cells infused could significantly predict platelet engraftment before or after 50 to achieve 100,000 platelets per microliter. Wilk's lambda for phospho-GSK-33 and HoxB4 and the number of CD34+ cells infused was significant (p=0.019) and the accuracy of classification into the 2 groups was 71%. Wilk's lambda for phospho-GSK-3β and MYSM1 and the number of CD34+ cells infused was significant (p=0.22) and the accuracy of classification into the 2 groups was 75%.

Engraftment was measured as the time after transplantation for the patient to achieve 100,000 platelets per microliter in the peripheral blood. This value is important because it indicates a significant level of platelet function.

Testing the Model: Method for Selection of Peripheral Blood Samples from Persons Pharmacologically Treated to Mobilize Bone Marrow Cells into the Peripheral Circulation for Therapeutic Transplantation

We assessed samples of peripheral blood from persons treated with various agents to mobilize bone marrow cells into the peripheral circulation. After the molecular expression data was obtained with investigators blinded to the clinical outcome data, engraftment time (days to 100,000 platelets per microliter) and the number of CD34⁺ cells infused were provided. Statistical analysis was accomplished with SPSS.

13 samples associated with fast engraftment of platelets (<50 days) and 11 associated with slow engraftment of platelets (>50 days).

Using linear discriminant analysis, we found that the expression levels of phospho-GSK-3β, NPM1, and the number of CD34⁺ cells infused could significantly predict prolonged time to platelet engraftment (100,000 platelets per microliter). Wilk's lambda for phospho-GSK-3β and the number of CD34⁺ cells infused was significant (p=0.009) and the accuracy of classification into the 2 groups was 75%. Additionally, NPM1 expression levels and the number of CD34⁺ cells infused gave a significant Wilk's lambda (p=0.04) and the accuracy of classification into the 2 groups was 75%.

We proposed a linear regression model of NPM1 expression levels and the number of CD34⁺ cells infused as independent factors predicting the day of achieving 20,000 platelets per microliter. This model was found to be significant (p=0.016) and both independent variables (NPM1 levels p=0.02; number of CD34⁺ cells infused p=0.02) were found to be significant. Thus, NPM1 expression levels was a significant independent factor predicting platelet engraftment.

Nucleophosmin 1 is a phosphorylated ribonucleoprotein mostly associated with the nucleolus. It binds to nucleic acids and is involved in the biogenesis of ribosomes. It has multiple functions including histone chaperone, DNA repair, endoribonuclease activity, and apoptosis inhibition. In a mouse model NPM1 was found to play a role in maintaining hematopoietic stem cell numbers and in preserving the functional integrity of the cells. The level of NPM1 expression directly affects repopulating ability in vivo but does not influence the fate commitment of the cells. Mutations of NPM1 have been found associated with a proportion of patients with acute myeloblastic leukemia.

Accordingly, we have successfully developed a model that significantly predicts engraftment time after transplantation of peripheral blood from persons pharmacologically treated to mobilize bone marrow cells into the peripheral circulation. The model includes the number of CD34⁺ cells infused and the expression level of nucleophosmin 1 (NPM1) in the CD34⁺ cells. This finding demonstrates the potency of a set of hematopoietic stem cells assessed by the expression level of a molecule in the cells prior to transplantation. So NPM-1, along with the number of CD34⁺ cells infused, can significantly predict engraftment time as indicated by the recovery of platelet numbers.

Although NPM1 was the molecule that provided the most definitive predictive power, there were several other molecules that performed similarly, including GSK-33, HoxB4, and MYSM1. The molecules were highly inter-correlated; they segregated to the same principal component. NPM1, GSK-3β, HoxB4, and MYSM1 are all part of the same cellular engine.

### Example 2

Hematopoietic stem/progenitor cell transplantation is an established therapeutic modality for a number of clinical circumstances. This procedure has two negative clinical outcomes that are preferable to avoid. Relapse of the neoplastic disease is an ominous sign for both allogeneic and autologous transplantation. For autologous transplantation it is the major cause of treatment failure. Relapse is less common in allogeneic transplantation but still it remains a frequent event. Additionally, graft-versus-host disease is also an undesirable sequela of hematopoietic stem/progenitor cell transplantation. In this case, the transplanted cells attack the host cells because they appear to be foreign.

Consequently, it would be valuable to predict these two negative clinical outcomes prior to the transplantation. In that way, different options can be sought in cases that analysis indicates is likely to result in either relapse or graft-versus-host disease.

We have analyzed 25 samples of frozen peripheral blood mononuclear cells from persons treated with plerixafor and G-CSF in order to mobilize their hematopoietic stem/progenitor cells from the bone marrow to the peripheral circulation. The cells were assessed for the expression levels of various molecules in the CD34+ hematopoietic stem/progenitor cell subset.

The samples had been used in autologous transplantation, and the clinical outcome data including relapse status were available. Expression levels of several molecules were significantly correlated with relapse status:

| | r = Pearson correlation | |
|---|---|---|
| Molecule | coefficient | p value |
| FLI1 (tech 1) | 0.66 | <0.001 |
| FLI1 (tech 2) | 0.56 | 0.003 |
| Musashi-2 | 0.61 | 0.001 |
| Profilin-1 | 0.53 | 0.007 |
| AML-1 | 0.57 | 0.003 |
| phospho-Akt(ser473) | 0.59 | 0.002 |
| DJ1 | 0.55 | 0.005 |

Linear discriminant analysis was used to assess the capacity of predicting relapse by expression level in CD34+ cells used in the subsequent transplantation. The results follow:

Including the expression levels of FLI-1 (tech 1)+Musashi-2+Profilin-1+AML-1+phospho-Akt(ser473) in the CD34+ cells collected prior to transplantation allowed for the correct classification of 92% of cases based on relapse after transplantation. The p value for this analysis is 0.009.

Including the expression levels of FLI-1 (tech 1)+Musashi-2+Profilin-1+AML-1 in the CD34+ cells collected prior to transplantation allowed for the correct classification of 88% of cases based on relapse after transplantation. The p value for this analysis is 0.005.

Thus, we have reduced to practice the capability of predicting an important clinical outcome, relapse after transplantation, by assessing the expression levels of certain molecules in CD34+ cells that were collected prior to transplantation.

The invention is, thus directed to the specific findings as indicated above and the more general principal of predicting eventual clinical outcome based on molecular expression levels in CD34+ cells prior to transplantation. More generally, the invention is directed to the prediction of clinical outcome by assessing molecular expression levels in cells of any kind in the transplanted inoculum.

All citations of our work in the description text are incorporated by reference for disclosing genes to which the methods of the invention can be applied.

1. A method to assess the ability of a sample of cells to achieve a desired clinical outcome that would result from transplanting the sample of cells into a subject, the method comprising assessing the level of expression of a desired gene product in cells in the sample to be transplanted and predicting the clinical outcome based on the measured level of expression in a desired number of individual cells, the level of expression and number of cells with that level having been previously correlated with a successful or unsuccessful clinical outcome.

2. The method of claim 1 in which the level of expression is compared to the mean or median expression level in about 20 or more samples of the same origin and type.

3. The method of claim 1 in which the cells are selected from the group consisting of hematopoietic-reconstituting cells, mesenchymal stem cells, dendritic cells, T lymphocytes, multipotent adult progenitor cells and neural stem cells or neural progenitor cells.

4. The method of claim 1 wherein the cells in which the level of expression is assessed are hematopoietic-reconstituting cells (hematopoietic stem cells and hematopoietic progenitor cells).

5. A method for assessing the capacity of a sample to therapeutically effect hematopoietic reconstitution in a subject, the method comprising: assessing CD34+ cells for enhanced expression of one or more of AML-1, MYSM1, Hif1α, Profilin-1, phospho-GSK-3beta, SKP2, cbx7, Bmi-1, TCF1, Musashi-2, or FLI1, in individual cells in the sample.

6. The method of claim 5 wherein the one or more of AML-1, MYSM1, Hif1a, Profilin-1, phospho-GSK-3beta, SKP2, cbx7, Bmi-1, TCF1, Musashi-2, or FLI1 are expressed at a level greater than the mean or median expression level in a sample of about 20 or more specimens of the same origin and type.

7. The method of claim 6 wherein the specimens are umbilical cord blood or mobilized peripheral blood.

8. A method to prepare a subject to donate blood for hematopoietic-reconstituting cell (HRC) transplantation, the method comprising obtaining a blood sample containing hematopoietic cells from the subject; determining number of CD34+ cells having enhanced expression of one or more of AML-1, MYSM1, Hif1a, Profilin-1, phospho-GSK-3beta, SKP2, cbx7, Bmi-1, TCF1, Musashi-2, or FLI1 in individual cells from the blood sample; and administering to the subject a mobilizing agent when the blood sample does not contain a desired therapeutically-effective amount of such CD34⁺ cells.

9. The method of claim 8 wherein the one or more of AML-1, MYSM1, Hif1a, Profilin-1, phospho-GSK-3beta, SKP2, cbx7, Bmi-1, TCF1, Musashi-2, or FLI1 are expressed at a level greater than the mean expression level in a sample of 20 or more specimens of the same origin and type.

10. The method of claim 8, further comprising the step of administering a mobilizing agent to the subject prior to the step of obtaining a blood sample.

11. A method for transplanting hematopoietic-reconstituting cells in a subject in need thereof, the method comprising administering to the subject nucleated blood cells comprising a therapeutically effective amount of CD34⁺ cells having enhanced expression of one or more of AML-1, MYSM1, Hif1a, Profilin-1, phospho-GSK-3beta, SKP2, cbx7, Bmi-1, TCF1, Musashi-2, or FLI1.

12. The method of claim 11 wherein the one or more of AML-1, MYSM1, Hif1a, Profilin-1, phospho-GSK-3beta, SKP2, cbx7, Bmi-1, TCF1, Musashi-2, or FLI1 are expressed at a level greater than the mean expression level in a sample of 20 or more specimens of the same origin and type.

13. The method of claim 11 wherein the CD34⁺ cells expressing the one or more of AML-1, MYSM1, Hif1a, Profilin-1, phospho-GSK-3beta, SKP2, cbx7, Bmi-1, TCF1, Musashi-2, or FLI1 are isolated.

14. The method of claim 11 wherein the subject has a disorder treatable by hematopoietic stem cell transplantation.

15. The method of claim 14 wherein the disorder is a hematopoietic deficiency or malignancy.

16. A method to identify a molecule, the expression of which is correlated with the hematopoietic reconstituting function, the method comprising assessing expression of a molecule in individual CD34⁺ cells in samples having different levels of potency and identifying molecules, the expression of which correlates with potency, by correlating differences in expression of such molecules with the potency of the different samples.

17. The method of claim 16 wherein greater potency is associated with an increase in expression.

18. The method of claim 16 wherein greater potency is associated with a decrease in expression.

19. The method of claim 16 wherein the samples that are compared are un-mobilized bone marrow, mobilized peripheral blood from healthy subjects, and umbilical cord blood.

20. The method of any of claims 1, 5, 6, or 11 wherein the expression level that is assayed is selected from the group consisting of RNA, protein, and post-translational modification.

21. A method to assess the potency of a sample for hematopoietic reconstitution function, the method comprising assessing the expression level of a molecule identified by the method in claim 20 in CD34⁺ cells in the sample.

22. The method of claim 16 wherein the different samples demonstrate varying degrees of the ability to provide a desired clinical outcome.


SEQUENCE LISTING (24 sequences)

SEQ ID NO: 1 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 2 - DNA, 1899 bp, Homo sapiens
gagcatcaccaacccacagccaaggcggcgctggcttttttttttttttttttaatctttaacaatttgaatatttgtttttacaaaggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactccccgaccaacatggcgccctccgcgcgcctggaggaggccgtgtggaggccctactgaggcgccaggcctggcccggctgggccccgcgggccgccgccttcgcctccgggcgcgcgggcctcctgtccgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagatt

SEQ ID NO: 3 - protein, 385 aa, Homo sapiens [sequence omitted]

SEQ ID NO: 4 - DNA, 1932 bp, Homo sapiens
ggcaacaacaaacgtgcacaggggagtgagggcagggcgctcgcagggggcacgcagggagggcccagggcgccagggaggccgcgccgggctaatccgaaggggctgcgaggtcaggctgtaaccgggtcaatgtgtggaatattggggggctcggctgcagacttggccaaatggacgggactattaaggaggctctgtcggtggtgagcgacgaccaatccctctttgactcagcgtacggagcggcagcccatctccccaaggccgacatgactgcctcggggagtcctgactacgggcagccccacaagatcaaccccctcccaccacagcaggagtggatcaatcagccagtgagggtcaacgtcaagcgggtgtatgaccacatgaatggatccaggcgccacgtgcctcattaaagagcagccttttatgctgggcttcacctgtacccaccctgagcggcagccgtggagccccatggccgcacgcagggcttgcgctggctggaggaggcacggtgcttgggagctgcaagaatggagggaggactggcaggcgagcgggcgagggagtctccggtggactgcagcgttagcaaatgcagcaagctggtgggcggaggcgagtccaaccccatgaactacaacagctatatggacgagaagaatggcccccctcctcccaacatgaccaccaacgagaggagagtcatcgtccccgcagacgccacactggtgacacaggagcatgtgaggcaatggctggagtgggccataaaggagtacagcttgatggagatcgacacatcctttttccagaacatggatggcaaggaactgtgtaaaatgaacaaggaggacttcctccgcgccaccaccctctacaacacggaagtgctgttgtcacacctcagttacctcagggaaagttcactgctggcctataatacaacctcccacaccgaccaatcctcacgattgagtgtcaaagaagacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctgggggcagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctccacggggggaatctaccccaaccccaacgtcccccgccatcctaacacccacgtgccttcacacttaggcagctactactagaagcttactcatcagtggccttctagctgaagcccatcctgcacacttactggatgctttggactcaacaggacatatgtggccttgaagggaagacaaaactggatgttctttcttgttggatagaacctttgtatttgtttctttaaaaacattttttttaatgttggtaacttttgcttcctctacctgaacaaagagatgaataatccatgggccagtatgccagttt

SEQ ID NO: 5 - protein, 735 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 6 - DNA, 3955 bp, Homo sapiens
gcgcgcgccggcctgggcaggcgagcgggcgcgctcccgccccctctcccctccccgcgcgcccgagcgcgcctccgcccttgcccgccccctgacgctgcctcagctcctcagtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggcaaaaaaaaaaaaaaaaaaaaaaa

SEQ ID NO: 7 - protein, 828 aa, Homo sapiens
MAAEEADVDIEGDVVAAAGAQPGSGENTASVLQKDHYLDSSWRTENGLIPWTLDNTISEENRAVIEKMLLEEEYYLSKKSQPEKVWLDQKEDDKKYMKSLQKTAKIMVHSPTKPASYSVKWTIEEKELFEQGLAKFGRRWTKISKLIGSRTVLQVKSYARQYFKNKVKCGLDKETPNQKTGHNLQVKNEDKGTKAWTPSCLRGRADPNLNAVKIEKLSDDEEVDITDEVDELSSQTPQKNSSSDLLLDFPNSKMHETNQGEFITSDSQEALFSKSSRGCLQNEKQDETLSSSEITLWTEKQSNGDKKSIELNDQKFNELIKNCNKHDGRGIIVDARQLPSPEPCEIQKNLNDNEMLFHSCQMVEESHEEEELKPPEQEIEIDRNIIQEEEKQAIPEFFEGRQAKTPERYLKIRNYILDQWEICKPKYLNKTSVRPGLKNCGDVNCIGRIHTYLELIGAINFGCEQAVYNRPQTVDKVRIRDRKDAVEAYQLAQRLQSMRTRRRRVRDPWGNWCDAKDLEGQTFEHLSAEELAKRREEEKGRPVKSLKVPRPTKSSFDPFQLIPCNFFSEEKQEPFQVKVASEALLIMDLHAHVSMAEVIGLLGGRYSEVDKVVEVCAAEPCNSLSTGLQCEMDPVSQTQASETLAVRGFSVIGWYHSHPAFDPNPSLRDIDTQAKYQSYFSRGGAKFIGMIVSPYNRNNPLPYSQITCLVISEEISPDGSYRLPYKFEVQQMLEEPQWGLVFEKTRWIIEKYRLSHSSVPMDKIFRRDSDLTCLQKLLECMRKTLSKVTNCFMAEEFLTEIENLFLSNYKSNQENGVTEENCTKELLM

SEQ ID NO: 8 - DNA, 7768 bp, Homo sapiens
gacggacggggtcaggtcccatcatggcggctgaagaggcggatgtggatatcgaaggggacgtggtagcggcggcgggggcacagccaggaagtggtgaaaatacagcatcagttttacaaaaagatcactatcttgattcatcttggagaacagagaatggccttattccttggaccttggataacaccatcagtgaagagaacagagctgttattgagaaaatgttgttggaagaagaatattatttatctaaaaaatcacaaccggaaaaagtctggcttgatcaaaaggaagatgataaaaaatacatgaagagtctgcagaaaacagcaaaaatcatggtacactctcctacaaaaccagccagttactcagtaaagtggacgatagaagaaaaagagctgtttgaacaagggctggctaaatttggccgaagatggaccaaaatttcaaagctaattggaagccgcactgttttacaagtgaagagttatgcaagacagtattttaaaaataaggtcaaatgcggtctggataaagaaacaccaaatcagaagaccggccataatcttcaagttaaaaatgaagataaagggacaaaggcatggacaccatcatgtttaaggggacgtgctgatcccaacttgaatgctgtaaaaattgaaaagttatctgatgatgaagaagtagacatcacagatgaggtggacgagttgtcttctcaaacaccccagaagaattctagcagtgatctcttgttagactttcctaatagtaaaatgcatgaaaccaatcaaggagaattcattacttctgacagccaggaagctctcttttctaagtcttccaggggctgtcttcaaaatgaaaagcaagatgaaacactttcaagctcagaaattacactgtggactgagaaacagagcaatggtgacaaaaaatcaattgaattaaatgaccagaaatttaatgaattgattaaaaactgcaacaagcatgatggaaggggaataatagttgatgccaggcagttgccttctccagagccttgtgaaattcagaaaaatttgaatgataatgaaatgctttttcattcttgccaaatggtagaggaaagccatgaggaagaagagcttaagccaccagaacaggaaatagaaatagatagaaatatcattcaagaagaagaaaaacaagcaattcctgagttttttgaggggcgccaagctaaaacaccagaacgctatttgaaaattagaaattatattttggatcaatgggagatatgcaaaccaaaatacttaaataagacctcagtacgtcctggcctgaagaactgtggagatgttaattgtattggacggattcatacatacctcgaattgataggagcaatcaattttggatgtgaacaggctgtgtataataggccacaaacagttgacaaagtacgaatcagagacagaaaagatgcagtagaagcataccaacttgcccagcgtctgcagtctatgcgtacaaggagacgtagggtccgagacccatggggaaactggtgtgatgcaaaggacttagaaggacaaacgtttgagcatctctctgctgaggagttggcaaaaagaagagaagaggaaaaaggcagacctgttaaatctttaaaagtgccaagaccaacaaaaagctcgtttgatcccttccaactgataccttgtaatttttttagtgaagaaaagcaggagccatttcaggtgaaagtggcttcagaagcacttttaataatggatttgcatgctcatgtttctatggcagaagtgattggtctgttaggaggaagatactcagaagttgataaagtagttgaagtctgtgcagcagaaccatgtaacagtctgagtacaggactacagtgtgagatggatcctgtatcacaaacacaggcctcagaaaccttggctgttagaggcttcagtgttattggatggtatcattctcatcctgcttttgatcctaatccttccttacgagatattgacacacaagctaaataccagagttacttctccagaggaggtgcaaagttcattgggatgattgttagtccctataatcgaaataatcccttaccatattctcagattacctgcctggttataagtgaggaaattagcccagatggctcttatcgcttaccttacaaatttgaagtacagcagatgttagaagaacctcagtggggattagtatttgaaaagacaagatggataatagaaaaatacaggctctcccatagcagcgtccccatggataaaatctttcgccgggattctgacctgacttgtttgcagaaacttttggagtgtatgaggaagactctgagcaaagtgaccaattgctttatggctgaagaattcttgactgaaatagaaaatttgttcctttccaattataaaagcaaccaagagaatggagtaaccgaagagaactgtacaaaggaattgttaatgtgattattttaaagttaagacattttaatcttgacacagtagatcttactttcaaagttataaacttgaagtgattgtagttaacattggcacagctttggtattttttctctatttcacaaaatccaaactttgccacataaatcatgtgaaaaggaagagatacaaatttgttttcatatagtgattatcagagtgttctgcaaaccagggtccaccacggtgttctagtcctttactgagcaatgctgtaggctgtgaaactaagcaacctggtcggctttactgttgtgttcagcttgggatttggactatgtctctaagtcatttcttccctgaactagtcatttatgttccattgtggtatctcctatttcatttaaaagcacttccattctttccatatattctgctgagatttgtttatggctataaatgagatatggatgggtggtcactgagataattaatatggtttagaactactgattaataaatctaacaggagaccaggagaggcatgacaagaaaatcttaaaaacaggaaaaacccaaaggagagaactaaattaccagtttctaatcatagatactatcttccatttcattgtatagcagtagtatcataagatatgccattttctcagctatctgaaaatttttaaccaattttacacacataaaataattagcttcaattgtgagtccacttacattagtaggtgtgataaaacctcatttaactggaacataggtaatcataactcaaactttttttctactgcctactactaggagaaagaggcaaatgataataaagctggaataaatcagagatttaatttttaaaacaagcctttcaggacatgttacatattcagcccagtttcatatgctttctgaatctataatggaggtcagtgaacttttttaatgtaaagggccagtttgtaaataattttggcttttcaggccacatatgatgtctatgagacattcttctttgtgtgtgtgtgtgtgtgtgtgtgtgtgtgtgtgtgttttacagccctttaaaatgataaaaaccattgttaactcacaggcctcataaaacataggctgttggctattattctccaacccttgatctagagaattttaaacattagaattttgttttacatttgttaagctttacgtcatcttcaaatgacaccaaaactcagaattatgatctgcatttaagccaattttaaaataaaataagcagtcaatgctgcctcatttattgaaattcagtagattttagtttatttcttagtagtcagagaattgatttatagctattcaccaaacattcttactaacgtttccttatatctaaatcagattgattctatattttgctttgtttgtaatcaatagccaatgaagaataaggagtattggaagaagcagtgagtcagctttaggtgacctggttcttgtctcaactttgtgatttggggagatacagtttactttccctgagccttaattcttgtgtaactgaagtctaacttaacttcttaatgtccttccagaactaaaattttattgttcattctaatcgttggactactcttgtgaagtttatattttgagttaatacagtttccaaattaacatacatttaaaaaaaaaagggtagtatttttccatttctctgtgtgcacttaggataaacagtgaagtatagcttataaaacaattagtttgagggctgagagtgtaagaggaactataccaaaagtcaggaaacctgagctcttaccctaactctgttaccaactttgctgtgtgaccttaatcaaaacacataacctggaccccatgctcctcaccagtaaatcaagggcctgaattttatggcttagtattcccttccaagctccgaagttgcgtgattctgtgaaaggcagtgattgattcctttgtactcatcttccttttgacctgtttcctcatgatatattggggaacaaatactaaaaagtgttttatactagctttcttgattgacatttccctataatactgatgaatttgggtgatggaaagtaatggaaattgttaaaagttctgctctcaaatctgagtctccttgccctgtgtgccaatgtttaaccatatttgctaatctaagccatattgcgaggatctcaaggatgatacttgtcaagagtttggggccttgagagcacagttttcaaaataatacttagtattttctgactatagagtacatttgtcttgtaaagtatattttaaaatacagagaagtgtgaagtacaaatatctcagtgctactatttaaaaaaacacaattagtatataaatccttccccatttttttgacagtataattttagtatgttaaacttggttacttctttctctcttcctccatcattttccctgcttcctttttcttttgtcatattcttaaaacaattttgcaaccacaggtacccacattgctggcactaccagccttgggctactaccgaggataatggagccagggccttgtagaagcacagaggaagtctagaactgagggggcctatgatacaggaagttcaggaggcagagtctcaaaggaacacctgcctttaataatgtttacagaagtggagaaaggactaaaggacttaaagacatgcaggtgtcttcatcctgattctgtctttgttattgcacaatgactataaaataatgacctaatttttttttttttctggcttggctaatggaaatcgacactagcgcttccccagtattagacttaatctaattccaaagtaacattcaagaaaagaaaaaccagtggaacaggatcagtaagctcagactctccatctccattattttagccttgaccattcttctctaatccttttaaattatcttcagtttccagttcaagttacattgcttcagttacagtttgtttgaccctgtattcagagctgcattatgattcttgtgggccctagatagttttgccatcttagaccccttcttccatacaaaattaaaagtgatattttacaattgtatctgtatgaaatgaatgtattaatattttagattaaaacattttgtttagaagttcagttttttcttatgaggctaaaaaaaattaaaacatttttgtgggcttctaaaaaactgtgggccttaggtactgtgcctaatggataagttggcattgcctgtattctgaagggcatcattggaaaaatagcagtcattattctgtccccttctcccaccaccacagtccttggagaagcctttgccactccatccaggagcactgcagtttccacggttatagatctatagacttgaacttacttccttcccaacctaacgaaaactcacaaggattacttgggaccattcatcctcaaaaaagataggaggcagtggtttgttttagtcttcagatatttttgttttggtcagtccctaatctgtagttttgaaactcgtgccacacttgaaagggaaacatcctactacctttctttcctctccatttcccacaaaccctttccttcatcatcactccctgcattcttacttctcagcatataaatacaagacccttctttctattatttgttgaaagctaggcctgtaaaatagagatggacagaccatgcatgtttccatgaagctatttaatgctgctcccagtcttctgtccataccattccttccctgcccctaggagtggggttgaggtctgtgacatgcagactgtaagcacagtcagaacctctgcagctctcaacccaacagaggagactgtggcttcaacatgccaacctgagcaggtctcctctgcattaccttcctcccatttctcttcctgggtcctgatgaggtgagctggaggaaccaatagtcaggatgccctggaattgatgttgctctgttttgtgctcacaaaacagccatgtaacacatgagcacctgagttgaagccatccagtcgcaagtcagcagacagcagttgatggaattctcagtagaaattcagtaagggtgcccaaattttattttttttttcacctatactctaccaatatatggtatttaggaaaacttcttttatccaagcacaaagaaaagcacacacatttttataatcatctgcttcatcagggtaaataaataatttaacaaatttgatcagcatctctagtttaccagtactttaatattagttttcttacaaattaatgtattttgcaatatttccagaaaatcactgtgggagaataatgtaatatactttaggatatatttcctgaccttttcttaaatggcttcatgtgctattcccccaaattctgctttctctattttcttttttatttaacctataattatactttctagtatattctgagtctatcttctaagccaccttacatttttcctggaacaagggagggaataaatagttgcctccactaaatcaggaaaaaataaaaatatcccaggggagctgtgattatgccaagagctctaaacagaagtttgagaaggtaaaaattaagttgtagtatctgagttgtttttattttcttcctttggtgtttatgaaggtattcataagaactttaatttcaggggaaaaaaatgcctgatttgctatttttgacatttcctcgtctcttaagaagtcagttaaatatgttttcatagtttatattcctgtttcatagattactgtgaaacatgtatttaaacctatgaattataaaatagtatttagattctagcgtgagttaaatagattagtcatatatcttttagatttgtggatttgacatgtaaattatgtgttgtgtataagtaagttagttactaaacatatggcatggttattgataaacttgttgctatttttttccaaatgctatcagtgtttgtggacttttaaaaattagtttgaattttggaatgttctgtgataaaatgtaatttcaactattttgtacatttaaatatgccatgttgtatatgtctgtatttaaaaatgttgtaaatacctgcattttaagaattatgaaagattttcctcaaaaatgacagaactctccatacttaattgtgacacattataagatatctgattttaagcttttggattttgttctaaaaattaagtttaaacatgctgaaaattccataaaaataaaattttgaaaataaagtga

SEQ ID NO: 9 - protein, 265 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 10 - DNA, 1256 bp, Homo sapiens
cggacgcgtgggcggacgcgtgggcggacgcgtgggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgccgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtaaaaaaaaaaaaaaa

SEQ ID NO: 11 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 12 - DNA, 1365 bp, Homo sapiens
cccgcagggtccacacgggtcgggccgggcgcgctcccgtgcagccggctccggccccgaccgccccatgcactcccggccccggcgcaggcgcaggcgcgggcacacgcgccgccgcccgccggtccttcccttcggcggaggtgggggaaggaggagtcatcccgtttaaccctgggctccccgaactctccttaatttgctaaatttgcagcttgctaattcctcctgctttctccttccttccttcttctggctcactccctgccccgataccaaagtctggtttatattcagtgcaaattggagcaaaccctacccttcacctctctcccgccaccccccatccttctgcattgctttccatcgaactctgcaaattttgcaatagggggagggatttttaaaattgcatttgcaaagttcggtgtctgggctggcgagtgggggagggagggaatggggagtaggccccgcccctaccgtcctttgcaaataaaaatctagcggggcggggggggggaggagcaggaagtggcggtgcgagggctgctgcacagcgagcggagccgcggtccggacggcagcgcgtgccccgagctctccgcctccccccgcccgccagccgaggcagctcgagcccagtccgcggccccagcagcagcgccgagagcagccccagtagcagcgccatggccgggtggaacgcctacatcgacaacctcatggcggacgggacctgtcaggacgcggccatcgtgggctacaaggactcgccctccgtctgggccgccgtccccgggaaaacgttcgtcaacatcacgccagctgaggtgggtgtcctggttggcaaagaccggtcaagtttttacgtgaatgggctgacacttgggggccagaaatgttcggtgatccgggactcactgctgcaggatggggaatttagcatggatcttcgtaccaagagcaccggtggggcccccaccttcaatgtcactgtcaccaagactgacaagacgctagtcctgctgatgggcaaagaaggtgtccacggtggtttgatcaacaagaaatgttatgaaatggcctcccaccttcggcgttcccagtactgacctcgtctgtcccttccccttcaccgctccccacagctttgcacccctttcctccccatacacacacaaaccattttattttttgggccattaccccataccccttattgctgccaaaaccacatgggctgggggccagggctggatggacagacacctccccctacccatatccctcccgtgtgtggttggaaaacttttgttttttggggttttttttttctgaataaaaaagattctactaacaagg

SEQ ID NO: 13 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 14 - DNA, 7134 bp, Homo sapiens
cgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaaaaaaaaaaaaaaaa

SEQ ID NO: 15 - protein, 424 aa, Homo sapiens
MHRKHLQEIPDLSSNVATSFTWGWDSSKTSELLSGMGVSALEKEEPDSENIPQELLSNLGHPESPPRKRLKSKGSDKDFVIVRRPKLNRENFPGVSWDSLPDELLLGIFSCLCLPELLKVSGVCKRWYRLASDESLWQTLDLTGKNLHPDVTGRLLSQGVIAFRCPRSFMDQPLAEHFSPFRVQHMDLSNSVIEVSTLHGILSQCSKLQNLSLEGLRLSDPIVNTLAKNSNLVRLNLSGCSGFSEFALQTLLSSCSRLDELNLSWCFDFTEKHVQVAVAHVSETITQLNLSGYRKNLQKSDLSTLVRRCPNLVHLDLSDSVMLKNDCFQEFFQLNYLQHLSLSRCYDIIPETLLELGEIPTLKTLQVFGIVPDGTLQLLKEALPHLQINCSHFTTIARPTIGNKKNQEIWGIKCRLTLQKPSCL

SEQ ID NO: 16 - DNA, 3520 bp, Homo sapiens
aattcccagcaggccttgggcctcagtgcggccgcgaagcagagcgggctgtagagccttgcgcgcgcagtggggatggaacgttgctaggcttagcgggtctggctgctgggggcccgagcagcacgctcggagccgccgcgcgccaaagcgggaatctgggaggcgagcagctctgcagttaatgcacgtattttaaactcccgggcctgcggacgctatgcacaggaagcacctccaggagattccagacctgagtagcaacgttgccaccagcttcacgtggggatgggattccagcaagacttctgaactgctgtcaggcatgggggtctccgccctggagaaagaggagcccgacagtgagaacatcccccaggaactgctctcaaacctgggccacccggagagccccccacggaaacggctgaagagcaaagggagtgacaaagactttgtgattgtccgcaggcctaagctaaatcgagagaactttccaggtgtttcatgggactcccttccggatgagctgctcttgggaatcttttcctgtctgtgcctccctgagctgctaaaggtctctggtgtttgtaagaggtggtatcgcctagcgtctgatgagtctctatggcagaccttagacctcacaggtaaaaatctgcacccggatgtgactggtcggttgctgtctcaaggggtgattgccttccgctgcccacgatcatttatggaccaaccattggctgaacatttcagcccttttcgtgtacagcacatggacctatcgaactcagttatagaagtgtccaccctccacggcatactgtctcagtgttccaagttgcagaatctaagcctggaaggcctgcggctttcggatcccattgtcaatactctcgcaaaaaactcaaatttagtgcgacttaacctttctgggtgttctggattctctgaatttgccctgcagactttgctaagcagctgttccagactggatgagctgaacctctcctggtgttttgatttcactgaaaagcatgtacaggtggctgttgcgcatgtgtcagagaccatcacccagctgaatcttagcggctacagaaagaatctccagaaatcagatctctctactttagttagaagatgccccaatcttgtccatctagacttaagtgatagtgtcatgctaaagaatgactgctttcaggaatttttccagctcaactacctccaacacctatcactcagtcggtgctatgatataatacctgaaactttacttgaacttggagaaattcccacactaaaaacactacaagtttttggaatcgtgccagatggtacccttcaactgttaaaggaagcccttcctcatctacagattaattgctcccatttcaccaccattgccaggccaactattggcaacaaaaagaaccaggagatatggggcatcaaatgccgactgacactgcaaaagcccagttgtctatgaagtatttattgcaggatggtgtctcttctttagaacagggaaaataggcaggaagcccaattgctggagtacttagctagttttattcttggttttccctttgccttcattctgcaagtatactagggagccatttgagagggaaaactatgaaatcttgctttttgaaatgattctaaaagcttctatcactgctttgctcttaagagccaaagttgtaggccttttgaaattttaggagagtgagcctataatttcaagataccttaaagagcaaaatttgagccacctcttccaagtgcccttcttactaagtctattcagaatcaagcttaaaaattaccaccagcaaacaatcttcatagcccatataacttttatctatttaattttatagtattgctttataagacagcttagaagaacaataagctatttgtattatgagctgaacaaaaagagaatcataggatagtagcgtctgaggccatcttttctaggaataggaaagagaaaaatgtatttgaattttgcctttagatttgaaattaggttaatagaaataagtaaccccatgtaattcaccttaaaacttaacaaaagaccaaacattacaaaacccagagatatagaatcaatataggatttgaaggcccagcagacagttttctatgacaggttaatctgaagtatcctgtaatgttcattaagttactgtgtttccagaatctaaattagatgagaaatataattgtggttttctaacttgataatcaaattatgttaacatgggtcctttagcttttaaaatgacttgctttgttttagaaaggtggtattaatccactctctattcttgaaaatttggatgggagaattctgaagttgcctgctgttttcctttagcgctgaggttcttaaggttacttttatattactctggaatcaagtattttaaattgtattttttttttaaatgatctctcagcaataattgtttgaaactatccatatataaggttatcagacctacagttccctaagaggaactgcatgttctcttcaatcagaaatatacagtagaagcaggtatatcttccatgcagtttcagtagtaagcactacttatacctacataagagttaaaatccagatgtgggaccttttgataccatcagtgatatatatttttttaaactggtacagagaagtgaaaagattaaattctacttctattttttttttttttttttgagacggagtctcgctctgtcaccaaggccggagtgcagtggtgcgatctcggctcactgcaagctccgcctcccaggttcacgtcattctcctgcctcagcctcccgactagctgggactacaggcgcccaccaccacgcccggctaatttttttgtatttttagtagagacggggtttcaccatgttagccaggatggtctcaatctcctgacctcatgatccgcccgtcttggcctcccaaagtgctgggattacaggcatgagcaactgcgcccagccaaattctacttcttaaaaatcacaaaaactagtttaaattgatgacttgttcgtatgttcaaaatgtaacaacaaaaaaagctaacaccagtcatttatattaactttttttttttaaatcaaaaattgttaatgttagaaacatactatgaagtgcctttatctgcttagacctaaggaagattttaaagttgggttgcacaggaaatgatgatgcttcaatttcttaatagttaaaaagtgctaaatactacttgaaattattgtttacagattagtgacaagagctggggttaggatccggttggactctgacatcggatgccctcaaacatacagaacttccaaactcaagtccagccataagctattttgccaacatgtcagagtaatctgtatttttgtatgtgatttctacttttatagacttgttttaaaacaataaaacacatttttataaaaatgagtgcttaaaaaaaaaaaaaaaaaa

SEQ ID NO: 17 - protein, 185 aa, Homo sapiens
MGKKQNKKKVEEVLEEEEEEYVVEKVLDRRVVKGKVEYLLKWKGFSDEDNTWEPEENLDCPDLIAEFLQSQKTAHETDKSEGGKRKADSDSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 18 - DNA, 558 bp, Homo sapiens
atggggaaaaaacaaaacaagaagaaagtggaggaggtgctagaagaggaggaagaggaatatgtggtggaaaaagttctcgaccgtcgagtggtaaagggcaaagtggagtacctcctaaagtggaagggattctcagatgaggacaacacatgggagccagaagagaacctggattgccccgacctcattgctgagtttctgcagtcacagaaaacagcacatgagacagataaatcagagggaggcaagcgcaaagctgattctgattctgaagataagggagaggagagcaaaccaaagaagaagaaagaagagtcagaaaagccacgaggctttgctcgaggtttggagccggagcggattattggagctacagactccagtggagagctcatgttcctgatgaaatggaaaaactctgatgaggctgacctggtccctgccaaggaagccaatgtcaagtgcccacaggttgtcatatccttctatgaggaaaggctgacgtggcattcctacccctcggaggatgatgacaaaaaagatgacaagaattaa

SEQ ID NO: 19 - protein, 326 aa, Homo sapiens
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKVNKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNAGELESDSGSDKANSPAGGIPSTSSCLPSPSTPVQSPHPQFPHISSTMNGTSNSPSGNHQSSFANRPRKSSVNGSSATSSG

SEQ ID NO: 20 - DNA, 3435 bp, Homo sapiens
acagcaactatgaaataatcgtagtatgagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcatgccattacagctttctagatgctaatacatgtgactatcgtccaatttgctttcttttgtagtgacattaaatttggctataaaagatggactacatgtgatactcctatggacgttaattgaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaacaagtgctttttatcttgaaattcaaccaacggaaagaatatgcatagaataatgcattctatgtagccatgtcactgtgaataacgatttcttgcatatttagccattttgattcctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatttataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaagattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcacccatcagttatttgtgagggtgtttattctatatgaatattgtttcatgtttgtatgggaaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaaagcacaatgtgcagaagttatggaaaacctatagaggattacaacaggtaaacgttaaagagaatacattgctgacttatagtgatgtggctaagaagtacatgctttgttgtaaaattgcttgaaagcccattgaaagatgtatctgtttatttacagtctttgaagtaaaagttaccaatgtttgccaataaaaa

SEQ ID NO: 21 - protein, 68 aa, Homo sapiens
MPQLDSGGGGAGGGDDLGAPNELLAFQDEGEEQDDKSRDSAAGPERDLAELKSSLVNESEGAAGGAGI

SEQ ID NO: 22 - DNA, 1362 bp, Homo sapiens
cgcgctccgcaaatctagatgtttcagcctggatcgagcgagggttagagggttagtcaggccagggtcaacttcagagtcatgggctccctaaatgcgacttctagggttgagttgctgtggacgagcgacccatgtcggaatcccgcgcccacgtggctgcccaaagttccgagtctccgggctgcaggttctagtcacggaaccgagttgggagagtcataggggctgggacttggaggatcggctgaggtccggtgctcttggctgtgttcgcggctcggagccgtcgcctgactgaggggcccgtcacagatgtgtgatgtataagctctgcacgcaacaggagctcaataaatgtgcgaaggggggtatacttatgatcgcactgtatgcggaggcggcctagaaggaagtccctgattggcacagggatggaggatggggcaagagccgcaacagcgccgcggagttccaacgctgccggttccctggggtacgagcacagcctcaagcagcctcaagccctaggagcccccacttcaaagcacagggcgcattggagcctgggcacgatacagttcacaccacggctgcgatggtaagccacgcccaagtcccaagggcctaggggacccccgccctccacagccggaggagaaacctgggcgcagaagcagggggaatatctggttgtaggtgagtaagcggggtcaggagttcccgttagagtctctgcgtttcgggagaagggtgatcattcccaggcttgtccgacgtctctctcagggtgcgctccggaagagcgagccctttaaggctatgccgagtgggcgcgtcccggcctctcccgggaagaggagagcgggggcgaacctgtgtccccgcccccggcccggcccgcccccagtgccggccccgcccccggcactcggccgccggcgcctttgatgttccgacccgccagctcgcggagccgcttctgccccgcgccctagcccgcgcctgcagcccgcccaggcggagtcagcccgcgctccgcccgccacgatccgggctcggaggttcggactccgggctcgccgccccccgggaccggctccgcgccccgcactcccggcgcccagcgccccgcgccccggcgggcggagcgcaccatgccgcagctggactccggcgggggcggcgcgggcggcggcgacgacctcggcgcgccgaacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcc

SEQ ID NO: 23 - protein, 328 aa, Homo sapiens
MEANGSQGTSGSANDSQHDPGKMFIGGLSWQTSPDSLRDYFSKFGEIRECMVMRDPTTKRSRGFGFVTFADPASVDKVLGQPHHELDSKTIDPKVAFPRRAQPKMVTRTKKIFVGGLSANTVVEDVKQYFEQFGKVEDAMLMFDKTTNRHRGFGFVTFENEDVVEKVCEIHFHEINNKMVECKKAQPKEVMFPPGTRGRARGLPYTMDAFMLGMGMLGYPNFVATYGRGYPGFAPSYGYQFPGFPAAAYGPVAAAAVAAARGSGSNPARPGGFPGANSPGPVADLYGPASQDSGVGNYISAASPQPGSGFGHGIAGPLIATAFTNGYH

SEQ ID NO: 24 - DNA, 1581 bp, Homo sapiens
ctcgccgctgccccggctccgccgctcgcagagagattcggaggagcccgggcgggggggaggaggagggggaggagggagcggagatctcggggctcggagccggccgccgctccgctccgatcgctgtggggcttggttttttgggggtgggggggcgggggggctcagatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttat